(19) United States
(12) Patent Application Publication
(10) Pub. No.: US 2024/0371469 A1
(43) Pub. Date: Nov. 7, 2024

(54) MACHINE LEARNING MODEL FOR RECALIBRATING GENOTYPE CALLS FROM EXISTING SEQUENCING DATA FILES

(71) Applicant: Illumina, Inc., San Diego, CA (US)

(72) Inventors: Rami Mehio, San Diego, CA (US); Gavin Derek Parnaby, Laguna Niguel, CA (US); Arun Visvanath, San Diego, CA (US); Zhuoyi Huang, Chula Vista, CA (US); Jacobus De Beer, San Diego, CA (US)

(21) Appl. No.: 18/654,914

(22) Filed: May 3, 2024

Related U.S. Application Data
(60) Provisional application No. 63/499,845, filed on May 3, 2023.

(51) Int. Cl. G16B 20/20; G16B 30/10; G16B 40/00

(52) U.S. Cl. CPC G16B 20/20; G16B 30/10; G16B 40/00

(57) ABSTRACT

This disclosure describes methods, non-transitory computer readable media, and systems that can utilize a machine learning model to recalibrate genotype calls (e.g., variant calls) of existing sequencing data files. For instance, the disclosed systems the disclosed systems can access one or more existing sequencing data files for a genomic sample, where the files include nucleotide-read data and genotype calls at particular genomic coordinate. From the one or more existing sequencing data files, the disclosed system extracts sequencing metrics for nucleotide reads or a particular genotype call at a particular genomic coordinate. By processing the extracted sequencing metrics, the systems further utilize a call-recalibration-machine-learning model to generate variant-call classifications indicating an accuracy of the particular genotype call. In some cases, the systems update or recalibrate the genotype call or quality-measuring sequencing metrics for the genotype call based on the variant-call classifications.

### CROSS-REFERENCE TO RELATED APPLICATIONS

This application claims priority to and the benefit of U.S. Provisional Patent Application No. 63/499,845, titled, “MACHINE LEARNING MODEL FOR RECALIBRATING GENOTYPE CALLS FROM EXISTING SEQUENCING DATA FILES,” filed on May 3, 2023, which is incorporated herein by reference in its entirety.

### BACKGROUND

In recent years, biotechnology firms and research institutions have improved hardware and software for sequencing nucleotides and determining variant calls for genomic samples. For instance, some existing nucleotide base sequencing platforms determine individual nucleotide bases within sequences by using conventional Sanger sequencing or by using sequencing-by-synthesis (SBS) methods. When using SBS, existing platforms can monitor many thousands to millions of nucleic acid polymers being synthesized in parallel to predict nucleotide base calls from a larger base call dataset. For instance, a camera in many SBS platforms captures images of irradiated fluorescent tags incorporated into oligonucleotides for determining the nucleotide base calls. After capturing such images, existing SBS platforms send base call data (or image-based data) to a computing device to apply sequencing data analysis software that determines a nucleotide base sequence for a nucleic acid polymer. Based on differences between the aligned nucleotide reads and the reference genome, existing systems can further utilize a variant caller to identify variants of a genomic sample, such as single nucleotide polymorphisms (SNPs), insertions or deletions (indels), and/or other structural variants, and genotype calls.

Despite these recent advances in sequencing and variant calling, existing nucleotide base sequencing platforms and sequencing data analysis software (together and hereinafter, existing sequencing systems) often determine or update models for determining variant calls that require considerable computing resources or execute variant callers that inaccurately determine nucleotide base calls or corresponding variant calls. For instance, some existing sequencing systems inefficiently expend considerable computing resources with overly complex models—often requiring considerable computer processing runtime—to accurately determine base calls or variant calls. To illustrate, some existing sequencing systems utilize variant callers with a deep learning architecture or some other neural network architecture that require extensive computational resources (e.g., computing time, processing power, and memory) to train and apply. For example, some existing sequencing systems utilize deep learning architectures that, even after training, take many hours across multiple computing devices to generate genotype calls for a single sample sequence.

In contrast to some deep learning architectures, some machine-learning-based variant callers have been developed that accurately determine variant calls by processing various features for both nucleotide reads and reference genome. But some such machine-learning-based variant callers are limited to a particular type of processor. For instance, some existing machine-learning-based variant callers can only be executed on a field programmable gate array (FPGA) or similar processing system. Due to such technical limitations, such existing machine-learning-based variant callers often cannot be executed on a remote server running a more mainstream processor, such as one or more of a central processing unit (CPU) or graphical processing unit (GPU).

As machine-learning-based variant callers have been updated or newly introduced, however, some existing sequencing systems re-sequence or regenerate variant call files for existing sequencing datasets to leverage the improved accuracy of the newer variant callers. While re-sequencing or regenerating variant call files using an updated version or newer machine-learning-based variant caller often improves variant-calling accuracy, such re-sequencing or regenerating approaches can double or triple the computational resources required to determine accurate variant calls for a given genomic sample. For example, for a database of over 100,000 genomic samples run on a previous version of a variant calling model, the computational runtime for a new version of a machine-learning-based variant caller to determine variant calls from read data could reach 2 million hours at 20 minutes per genomic sample.

Despite the computing time and other resources required to execute a machine-learning-based variant caller on previously called genomic samples, some existing sequencing systems expend such computing resources because the variant-calling accuracy of such systems carry significant diagnostic or research consequences. In some circumstances, for instance, existing sequencing systems apply a variant caller that inaccurately identifies excessive numbers of false negative variant calls. To illustrate, existing sequencing systems sometimes determine a genomic coordinate exhibits a homozygous reference genotype (and therefore not include a variant) when, in fact, the coordinate includes a variant. Indeed, existing variant callers achieve a certain level of accuracy but, due to their limitations, still leave room for improvement in recovering false negative variant calls. To illustrate the impact of such inaccuracy, a variant call identifying a particular single nucleotide polymorphism (SNP) in the hemoglobin beta (HBB) gene can have significant implications. When a variant caller identifies an SNP at rs344 on chromosome 11, for instance, the variant caller can either correctly identify the genetic cause of sickle cell anemia or miss the cause of the disease. As a further example, a variant call that correctly or incorrectly identifies the deletion of one or more copies of hemoglobin subunit alpha 1 (HbA1) or hemoglobin subunit alpha 2 (HbA2) genes can result in either correctly identifying a genetic cause of an inherited blood disorder or miss the gene deletion entirely.

As a contributing factor to the aforementioned inaccuracies, many existing sequencing systems leverage only limited sets of data in determining nucleotide base calls. For instance, existing sequencing systems frequently rely exclusively on information extracted directly from nucleotide reads of a sample sequence, such as read depth, mismatch counts, sequence alignment scores, and mapping quality, to determine nucleotide base calls. While sequence information from nucleotide reads can provide valuable insight for determining nucleotide base calls, existing sequencing systems that solely rely on these data can underperform in accurately determining nucleotide base calls, including variant calls. Indeed, some existing sequencing systems that rely on raw sequence data incorrectly determine SNPs, indels, or other variants in a genomic sample sequence in comparison to more complex models. Indeed, existing sequencing systems frequently identify false negative variants or false positive variants in the Truth Challenges of the U.S. Food and Drug Administration (FDA).

### SUMMARY

This disclosure describes embodiments of methods, non-transitory computer readable media, and systems that can utilize a machine learning model to recalibrate genotype calls (e.g., variant calls) or variant quality metrics of existing sequencing data files. As described below, the disclosed systems can access one or more existing sequencing data files for a genomic sample, where such existing files include nucleotide-read data and genotype calls at particular genomic coordinates. From such an existing sequencing data file, the disclosed system extracts sequencing metrics for nucleotide reads or particular genotype calls at particular genomic coordinates. By processing the extracted sequencing metrics, the systems further utilize a call-recalibration-machine-learning model to generate variant-call classifications indicating an accuracy of a particular genotype call. Based on the variant-call classifications, in some cases, the systems update or recalibrate the particular genotype call or quality-measuring sequencing metrics corresponding to the particular genotype call. After recalibrating the particular genotype call and other genotype calls for the genomic sample, the disclosed systems can output an updated or recalibrated sequencing data file for the genomic sample, such as an updated variant call file.

By extracting sequencing metrics for genotype calls from existing sequencing data files—and utilizing a call-recalibration-machine-learning model to generate variant-call classifications to updated previously generated genotype calls or corresponding metrics—the disclosed systems can improve accuracy, efficiency, and speed over existing sequencing systems. Significantly, the disclosed systems can also avoid the computational expense of re-running an entire machine-learning-based variant call model on sequencing data for a genomic sample to determine updated genotype calls or updated quality-measuring sequencing metrics for the genomic sample, as some existing sequencing systems must do.

### BRIEF DESCRIPTION OF THE DRAWINGS

The detailed description refers to the drawings briefly described below.

FIG. 1 illustrates a block diagram of a sequencing system including a call recalibration system in accordance with one or more embodiments.

FIG. 2A illustrates an overview of (i) an original call generation model processing base call data for a genomic sample to produce original sequencing data files and (ii) an updated call generation model re-processing the base call data through a call-recalibration machine-learning model for the genomic sample to generate updated sequencing data files in accordance with one or more embodiments.

FIG. 2B illustrates an overview of the call recalibration system using a call-recalibration-machine-learning model to analyze information from the original sequencing data files to generate recalibrated sequencing data files in accordance with one or more embodiments.

FIG. 3 illustrates the call recalibration system receiving existing sequencing data files, extracting sequencing metrics therefrom, generating variant-call classifications based on the extracted sequencing metrics, and generating a recalibrated sequencing data file (e.g., an updated variant call file) based on the variant-call classifications in accordance with one or more embodiments.

FIGS. 4A-4C illustrate the call recalibration system identifying or extracting sequencing metrics from existing sequencing data files or external sources and generating variant-call classifications in accordance with one or more embodiments.

FIGS. 5A-5C illustrate the call recalibration system generating variant-call classifications (e.g., genotype probabilities), generating corresponding recalibrated genotype calls or recalibrated sequencing metrics utilizing a call-recalibration-machine-learning model, and generating a recalibrated genotype-call data file comprising the updated genotype call based on such classifications in accordance with one or more embodiments.

FIG. 6 illustrates an example process for the call recalibration system training a call-recalibration-machine-learning model in accordance with one or more embodiments.

FIG. 7 illustrates a table describing compute nodes and runtimes for reprocessing base call data for genomic samples with an updated call-generation machine-learning model versus recalibrating genotype calls for the genomic samples using a call-recalibration-machine-learning model in accordance with one or more embodiments.

FIGS. 8A-8B illustrate graphs of false positives and false negatives (for SNPs or indels) comparing results of re-processing sequencing data with existing call-generation models and results of recalibrating existing genotype calls using the call-recalibration-machine-learning model in accordance with one or more embodiments.

FIG. 9 illustrates a flowchart of a series of acts for generating a recalibrated sequencing data file in accordance with one or more embodiments.

FIG. 10 illustrates a block diagram of an example computing device for implementing one or more embodiments of the present disclosure.

### DETAILED DESCRIPTION

This disclosure describes embodiments of a call recalibration system that recalibrates genotype calls (e.g., variant calls) or corresponding sequencing metrics for a sample nucleotide sequence utilizing a call-recalibration-machine-learning model. To recalibrate such genotype calls or corresponding metrics, the call recalibration system accesses one or more existing sequencing data files for a genomic sample. Such sequencing data files may include, for instance, a genotype-call data file, such as a variant call format (VCF) file, and/or an alignment data file, such as a binary alignment map (BAM) file or a compressed reference-oriented alignment map (CRAM) file). By processing sequencing metrics extracted from the existing sequencing data file through a call-recalibration-machine-learning model, the call recalibration system can generate variant-call classifications or predictions for confirming, recalibrating, or modifying a genotype call previously generated by a call generation model. Based on the variant-call classifications or predictions, the call recalibration system can also confirm or update various sequencing metrics of the previously generated genotype calls, such as a call quality, a genotype associated with the call, a genotype quality associated with the genotype, Phred-scaled Likelihood (PL), and/or other metrics with corresponding fields. By utilizing the call-recalibration-machine-learning model to update sequencing metrics, the call recalibration system can improve the confidence or reliability of updated genotype calls or confirmed genotype calls at particular genomic coordinates.

As mentioned above, in some embodiments, the call recalibration system identifies sequencing metrics for previously generated genotype calls. For instance, the call recalibration system extracts or determines sequencing metrics for a sample nucleotide sequence from one or more existing sequencing data files. To elaborate, in certain implementations, the call recalibration system extracts or determines, from one or more existing sequencing data files, different types of sequencing metrics associated with different sources. For example, the call recalibration system extracts or determines read-based sequencing metrics including metrics derived from nucleotide reads of the sample nucleotide sequence. In some embodiments, for example, the call recalibration system extracts read-based sequencing metrics from sequencing data file(s) that include nucleotide reads of the sample nucleotide sequence, such as an alignment data file (e.g., a binary alignment map (BAM) file or a compressed reference-oriented alignment map (CRAM) file).

Further, in some embodiments, the call recalibration system extracts or determines call-model-generated sequencing metrics generated via a variant caller or other call generation model, such as variables internal to the call recalibration system that are not accessible to other systems or parties (e.g., proprietary quality scores, base contexts, read filtering, proprietary hypothesis scores, and other metrics). In some implementations, the call recalibration system extracts at least some of the call-model-generated sequencing metrics from a genotype-call data file, such as, a variant call format (VCF) file or a genomic variant call format (gVCF) file.

Beyond read-based or call-model-generated sequencing metrics, in some implementations, the call recalibration system derives or re-constructs one or more of sequencing metrics, such as by reconstructing one or more call-model-generated sequencing metrics not expressly written to (stored within) the sequencing data file(s), from other information stored within the sequencing data file(s). Indeed, in some cases, the call recalibration system determines call-model-generated sequencing metrics in the form of variant calling sequencing metrics and mapping-and-alignment sequencing metrics, where some such metrics are derived or otherwise determined from one or more existing sequencing data files of various formats. For example, in some embodiments, the call recalibration system estimates alignment of each read in a sequencing data file (e.g., an alignment data file) to a reference genome utilizing a Concise Idiosyncratic Gapped Alignment Report (CIGAR), instead of depending on the hidden Markov model (HMM) score for each read.

In addition, in some embodiments, the call recalibration system extracts or determines externally sourced sequencing metrics identified from one or more external databases that indicate various nucleotide attributes, mapping challenges, and genomic sequences associated with sequencing biases. Alternatively or additionally, the call recalibration system extracts or determines externally sourced sequencing metrics stored within existing sequencing data files.

By processing the extracted sequencing metrics or reconstructed sequencing metrics, in certain implementations, the call recalibration system generates a set of predicted classifications upon which the system can modify or improve a given genotype call or fields associated with the given genotype call. More specifically, in some embodiments, the call recalibration system utilizes a call-recalibration-machine-learning model to generate, from the sequencing metrics, a set of variant-call classifications that impact or reflect the accuracy of identifying a variant at a particular genomic coordinate. Depending on the type of genomic coordinate, the call recalibration system can utilize a particularly trained version of a call-recalibration-machine-learning model to, for example, generate (i) variant-call classifications for multiallelic coordinates that differ from (ii) certain variant-call classifications from a different version of the call-recalibration-machine-learning model for haploid coordinates or would-be-false homozygous reference coordinates.

When generating variant-call classifications for a multiallelic genomic coordinate with an existing genotype call, for instance, the call recalibration system can utilize the call-recalibration-machine-learning model to generate a set of variant-call classifications including: (i) a reference probability that the genotype call comprises a homozygous reference genotype at the multiallelic genomic coordinate, (ii) a zygosity-error probability that the genotype call comprises a genotype-zygosity error at the multiallelic genomic coordinate, and (iii) a true-positive variant probability that a genotype call constitute a true positive variant at the multiallelic genomic coordinate. As another example, for a haploid genomic coordinate with an existing genotype call, the call recalibration system can utilize the call-recalibration-machine-learning model to generate a set of variant-call classifications including: (i) a first genotype probability of a first genotype at the genomic coordinate and (ii) a second genotype probability of a second genotype at the genomic coordinate. Further, for would-be homozygous reference coordinates, the call recalibration system can utilize the call-recalibration-machine-learning model to generate a set of variant-call classifications including: (i) a false-positive probability that a genotype call is a false positive variant, (ii) a zygosity-error probability that the genotype call comprises a genotype-zygosity error (e.g., a probability of identifying a correct alt allele but with a genotype-zygosity error—e.g., 0/1 instead of 1/1 or 1/1 instead of 0/1—or a probability of incorrectly identifying a genotype of a nucleotide base call), and (iii) a true-positive probability (e.g., homozygous alternate classification indicating a probability that a genotype call comprises a true positive variant).

Based on the variant-call classifications, the call recalibration system can confirm, modify, or update genotype calls or sequencing metrics corresponding to one or more genotype calls for a genomic coordinate (e.g., a variant call or a non-variant call). For example, the call recalibration system utilizes the variant-call classifications to update genotype data fields within a genotype-call data file (e.g., a variant call format file or other base call output file) that indicates or represents an updated genotype call with improved accuracy. As indicated above, in certain implementations, the call recalibration system updates genotype calls for specific genomic coordinates, such as multiallelic genomic coordinates, haploid genomic coordinates, and/or would-be falsely identified homozygous reference coordinates (e.g., genomic coordinates that were previously falsely identified by a variant caller to exhibit homozygous reference genotypes).

As a further example, in some embodiments, the call recalibration system utilizes (i) sequencing metrics extracted or determined from one or more existing sequencing data files and (ii) the call-recalibration-machine-learning model to modify data fields corresponding to an existing variant call file (or other genotype-call data file) for the genotype call. For instance, the call recalibration system updates one or more of a base-call-quality metric, a genotype-probability metric, a genotype-likelihood metric, or a genotype-quality metric for the genotype call in corresponding fields of a VCF or other sequencing data file. When the call recalibration system determines that a modified base-call-quality metric (e.g., Q score) or other confidence scores fails to satisfy a threshold, the call recalibration system can annotate the genotype call within the recalibrated VCF or other recalibrated sequencing data file to indicate the modified metric or score falls below a base-call-quality threshold or other metric or score threshold.

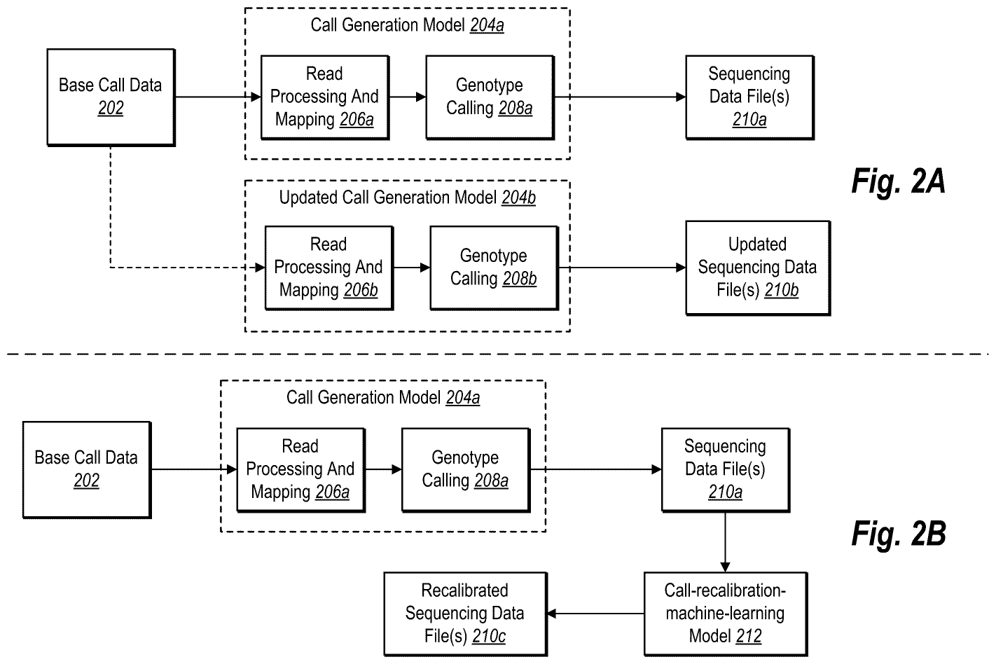

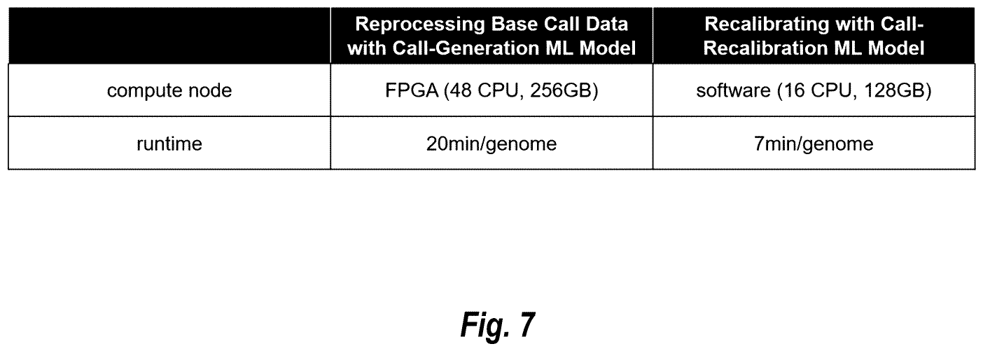

As suggested above, the call recalibration system provides several advantages, benefits, and/or improvements over existing sequencing systems, including variant callers and other sequencing data analysis software. For instance, the call recalibration system confirms or modifies genotype calls (or corresponding sequencing metrics) with less computational runtime than existing sequencing systems implementing a machine-learning-based variant caller. By extracting sequencing metrics from existing sequencing data files to analyze genotype calls and associated sequencing data, for example, the call recalibration system significantly improves processing runtimes relative to existing sequencing systems that re-analyze nucleotide-read data utilizing a new or updated call generation machine learning model. In some implementations, for example, the call recalibration system exhibits a 65% reduction in runtime per genomic sample sequencing compared to re-analyzing corresponding sequencing data with an existing call generation machine learning model. This disclosure further illustrates such improved computational runtime below with respect to at least FIGS. 2A-2B and 7.

The call recalibration system's improved efficiency and speed is particularly evident relative to machine-learning-based variant callers that employ deep learning architectures. As noted above, some existing sequencing systems utilize computationally expensive, slow neural network architectures (e.g., deep learning architectures such as convolutional neural networks) that require many hours (e.g., 5-8 hours with multiple processors executing on a server) and large amounts of computational resources to even implement and generate a file with variant calls from a sequencing run. Such deep learning architectures can further require several days (or weeks) to train. Conversely, the call recalibration system utilizes a comparatively lightweight, fast architecture for call-recalibration-machine-learning model. In contrast to the many hours across multiple processors required by existing sequencing systems, the call recalibration system, in many cases, requires under 10 minutes of runtime on general-purpose CPUs to recalibrate nucleotide base calls for a sample nucleotide sequence. Thus, the call recalibration system is far faster and less computationally expensive than many deep learning approaches to variant calling.

In addition to expedited computer processing, in some embodiments, the call recalibration system increases the processing flexibility with which a sequencing system can determine, modify, or update genotype calls or corresponding sequencing metrics using a machine-learning model. As indicated above, some existing machine-learning-based variant callers, for instance, run exclusively on a field programmable gate array (FPGA) or other hardware accelerator. By contrast, in one or more embodiments, the call-recalibration-machine-learning model of the call recalibration system can run on a general-purpose processing unit, such as but not limited to, one or more of a central processing unit (CPU) or a graphical processing unit (GPU). By training a call-recalibration-machine-learning model to modify and/or update genotype calls in existing sequencing data files, the call recalibration system can be implemented with significantly less computing resources compared to existing sequencing systems that utilize call generation machine learning models to generate or re-generate genotype calls. Accordingly, the call recalibration system can also be implemented with fewer processing cores and less processing memory. In one or more implementations, for example, the call recalibration system has exhibited the faster runtimes discussed above whilst requiring one third of the processing cores and half of the processing memory compared to re-analyzing with an existing machine-learning-based variant caller, resulting in reduced costs. This disclosure further illustrates such improved processing flexibility below with respect to at least FIG. 7.

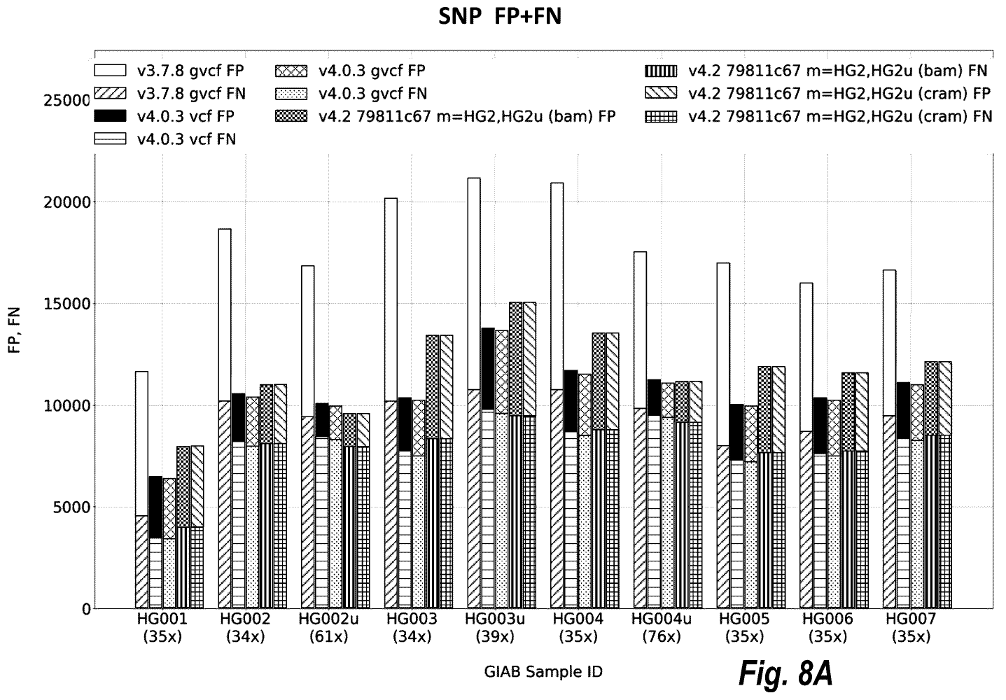

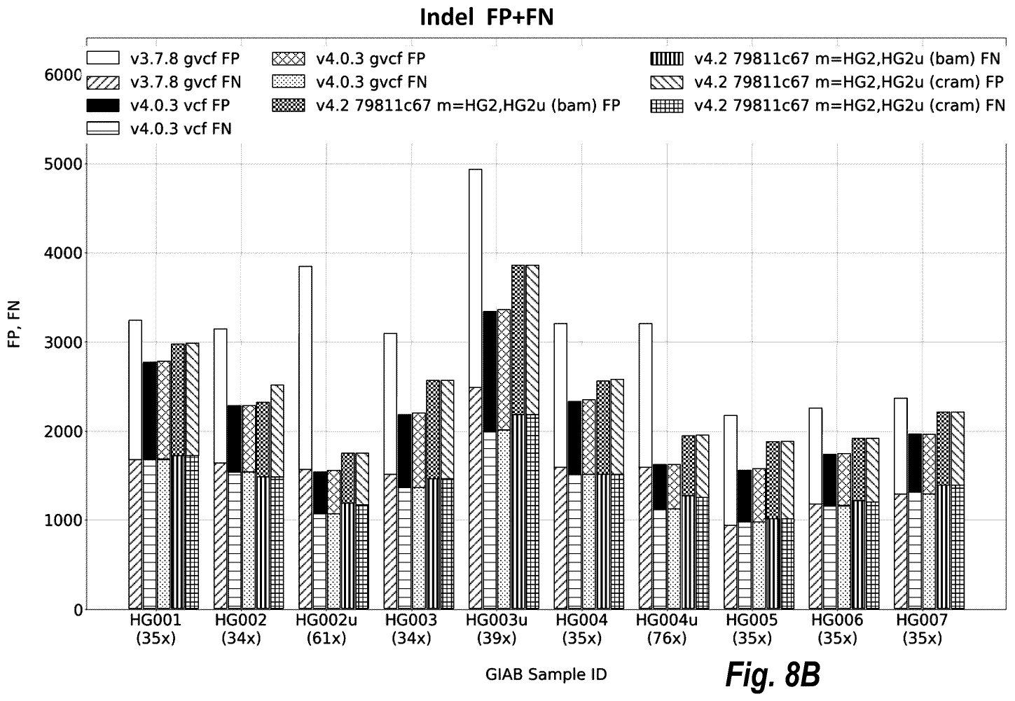

Beyond improved runtime and flexibility, the call recalibration system improves the accuracy of genotype calls or corresponding sequencing metrics from existing sequencing data files for genomic samples. In one or more embodiments, for example, the call recalibration system can utilize a call-recalibration-machine-learning model to update genotype calls or a corresponding base-call-quality metric, a genotype-probability metric, a genotype-likelihood metric, or a genotype-quality metric for the genotype call, based on sequencing metrics extracted from one or more existing sequencing data files. By utilizing a call-recalibration-machine-learning model to update genotype calls based on extracted sequencing metrics, the call recalibration system can improve the accuracy of genotype calls at biallelic genomic coordinates, multiallelic genomic coordinates, or haploid coordinates. The call recalibration system can likewise recover (i.e., correct) false negative variant calls and false positive variant calls that have been reported in existing sequencing data files. This disclosure further illustrates such improved accuracy below with respect to at least FIGS. 8A-8B.

As suggested by the foregoing discussion, this disclosure utilizes a variety of terms to describe features and benefits of the call recalibration system. Additional detail is hereafter provided regarding the meaning of these terms as used in this disclosure. As used in this disclosure, for instance, the term “sample nucleotide sequence” or “sample sequence” refers to a sequence of nucleotides isolated or extracted from a sample organism (or a copy of such an isolated or extracted sequence). In particular, a sample nucleotide sequence includes a segment of a nucleic acid polymer that is isolated or extracted from a sample organism and composed of nitrogenous heterocyclic bases. For example, a sample nucleotide sequence can include a segment of deoxyribonucleic acid (DNA), ribonucleic acid (RNA), or other polymeric forms of nucleic acids or chimeric or hybrid forms of nucleic acids noted below. More specifically, in some cases, the sample nucleotide sequence is found in a sample prepared or isolated by a kit and received by a sequencing device.

Relatedly, as used herein, the term “genomic sample” refers to a target genome or portion of a genome undergoing an assay or sequencing. For example, a genomic sample includes one or more sequences of nucleotides isolated or extracted from a sample organism (or a copy of such an isolated or extracted sequence). In particular, a genomic sample includes a full genome that is isolated or extracted (in whole or in part) from a sample organism and composed of nitrogenous heterocyclic bases. A genomic sample can include a segment of deoxyribonucleic acid (DNA), ribonucleic acid (RNA), or other polymeric forms of nucleic acids or chimeric or hybrid forms of nucleic acids noted below. In some cases, the genomic sample is found in a sample prepared or isolated by a kit and received by a sequencing device.

As further used herein, the term “genotype call” refers to a determination or prediction of a particular genotype of a genomic sample at a genomic locus. In particular, a genotype call can include a prediction of a particular genotype of a genomic sample with respect to a reference genome or a reference sequence at a genomic coordinate or a genomic region. For instance, in some cases, a genotype call includes a determination or prediction that a genomic sample comprises both a nucleobase and a complementary nucleobase at a genomic coordinate that is either homozygous or heterozygous for a reference base or a variant (e.g., homozygous reference bases represented as 0|0 or heterozygous for a variant on a particular strand represented as 0|1). Accordingly, a genotype call can include a prediction of a variant or reference base for one or more alleles of a genomic sample and indicate zygosity with respect to a variant or reference base. A genotype call is often determined for a genomic coordinate or genomic region at which an SNP, insertion, deletion, or other variant has been identified for a population of organisms.

As further used herein, the term “nucleotide base call” (or simply “base call”) refers to a determination or prediction of a particular nucleobase (or nucleobase pair) for an oligonucleotide (e.g., nucleotide read) during a sequencing cycle or for a genomic coordinate of a sample genome. In particular, a nucleobase call can indicate (i) a determination or prediction of the type of nucleobase that has been incorporated within an oligonucleotide on a nucleotide-sample slide (e.g., read-based nucleobase calls) or (ii) a determination or prediction of the type of nucleobase that is present at a genomic coordinate or region within a genome, including a variant call or a non-variant call in a digital output file. In some cases, for a nucleotide read, a nucleobase call includes a determination or a prediction of a nucleobase based on intensity values resulting from fluorescent-tagged nucleotides added to an oligonucleotide of a nucleotide-sample slide (e.g., in a cluster of a flow cell). Alternatively, a nucleobase call includes a determination or a prediction of a nucleobase from chromatogram peaks or electrical current changes resulting from nucleotides passing through a nanopore of a nucleotide-sample slide. By contrast, a nucleobase call can also include a final prediction of a nucleobase at a genomic coordinate of a sample genome for a variant call file (VCF) or another base-call-output file based on nucleotide reads corresponding to the genomic coordinate. Accordingly, a nucleobase call can include a base call corresponding to a genomic coordinate and a reference genome, such as an indication of a variant or a non-variant at a particular location corresponding to the reference genome. Indeed, a nucleobase call can refer to a variant call, including but not limited to, a single nucleotide variant (SNV), an insertion or a deletion (indel), or base call that is part of a structural variant. As suggested above, a single nucleobase call can be an adenine (A) call, a cytosine (C) call, a guanine (G) call, a thymine (T) call, or an uracil (U) call.

Relatedly, as used herein, the term “nucleotide read” refers to an inferred sequence of one or more nucleotide bases (or nucleobase pairs) from all or part of a sample nucleotide sequence (e.g., a sample genomic sequence, complementary DNA). In particular, a nucleotide read includes a determined or predicted sequence of nucleobase calls for a nucleotide fragment (or group of monoclonal nucleotide fragments) from a sequencing library corresponding to a genomic sample. For example, in some embodiments, the call recalibration system determines a nucleotide read by generating nucleobase calls for nucleobases passed through a nanopore of a nucleotide-sample slide, determined via fluorescent tagging, or determined from a well in a flow cell. In some cases, a nucleotide read can refer to a particular type of read, such as a nucleotide read synthesized from sample library fragments that are shorter than a threshold number of nucleobases (e.g., SBS reads). In these or other cases, another type of nucleotide read can refer to (i) assembled nucleotide reads that have been assembled from shorter nucleotide reads to form a contiguous sequence (e.g., assembled nucleotide reads) satisfying a threshold number of nucleobases, (ii) circular consensus sequencing (CCS) reads satisfying the threshold number of nucleobases, or (iii) nanopore long reads satisfying the threshold number of nucleobases.

As noted above, in some embodiments, the call recalibration system determines sequencing metrics for nucleotide base calls of nucleotide reads. As used herein, the term “sequencing metric” refers to a quantitative measurement or score indicating a degree to which an individual nucleotide base call (or a sequence of nucleotide base calls) aligns, compares, or quantifies with respect to a genomic coordinate or genomic region of a reference genome, with respect to nucleotide base calls from nucleotide reads, or with respect to external genomic sequencing or genomic structure. For instance, a sequencing metric includes a quantitative measurement or score indicating a degree to which (i) individual nucleotide base calls align, map, or cover a genomic coordinate or reference base of a reference genome; (ii) nucleotide base calls compare to reference or alternative nucleotide reads in terms of mapping, mismatch, base call quality, or other raw sequencing metrics; or (iii) genomic coordinates or regions corresponding to nucleotide base calls demonstrate mappability, repetitive base call content, DNA structure, or other generalized metrics.

Along these lines, the call recalibration system determines various types of sequencing metrics from different sources, such as read-based sequencing metrics, externally sourced sequencing metrics, and call-model-generated sequencing metrics. As used herein, the term “read-based sequencing metrics” refers to sequencing metrics derived from nucleotide reads of a sample nucleotide sequence. For example, read-based sequencing metrics include sequencing metrics determined by applying statistical tests to detect differences between a reference sequence and nucleotide reads. In some embodiments, read-based sequencing metrics can include a comparative-mapping-quality-distribution metric that indicates a comparison between mapping qualities or a comparative-mismatch-count metric that indicates a comparison between mismatch counts. In some cases, read-based sequencing metrics can correspond to genotype calls generated from different read types, such as assembled nucleotide reads and/or SBS reads.

By contrast, “externally sourced sequencing metrics” refer to sequencing metrics identified or obtained from one or more external databases. For example, externally sourced sequencing metrics include metrics relating to mappability of nucleotides, replication timing, or DNA structure that are available outside of the call recalibration system.

Further, the term “call-model-generated sequencing metrics” refers to internal, model-specific sequencing metrics generated or extracted by a call generation model. For example, call-model-generated sequencing metrics include variant calling sequencing metrics extracted or determined via variant caller components of a call generation model and mapping-and-alignment sequencing metrics extracted or determined via mapping-and-alignment components of a call generation model. As indicated above, call-model-generated sequencing metrics can include alignment metrics that quantify a degree to which nucleotide reads align with genomic coordinates of a reference genome or other example nucleic acid sequence, such as deletion-size metrics or mapping-quality metrics. Further, call-model-generated sequencing metrics can include depth metrics that quantify the depth of nucleobase calls for nucleotide reads at genomic coordinates of a reference genome or other example nucleic acid sequence, such as forward-reverse-depth metrics or normalized-depth metrics. Call-model-generated sequencing metrics can also include call-quality metrics that quantify a quality or accuracy of nucleobase calls, such as nucleobase-call-quality metrics, callability metrics, or somatic-quality metrics.

As used herein, the term “base-call-quality metric” refers to a specific score or other measurement indicating an accuracy of a nucleobase call. In particular, a base-call-quality metric comprises a value indicating a likelihood that one or more predicted nucleobase calls for a genomic coordinate contain errors. For example, in certain implementations, a base-call-quality metric can comprise a Q score (e.g., a PHil's Read EDitor (PHRED) quality score) predicting the error probability of any given nucleobase call. To illustrate, a quality score (or Q score) may indicate that a probability of an incorrect nucleobase call at a genomic coordinate is equal to 1 in 100 for a Q20 score, 1 in 1,000 for a Q30 score, 1 in 10,000 for a Q40 score, etc.

Relatedly, in some embodiments, the call recalibration system can generate sequencing metrics through modifying or updating previous metrics. Such “re-engineered sequencing metrics” can refer to sequencing metrics that have been updated, modified, augmented, refined, or re-engineered to measure or compare nucleobase calls (e.g., nucleobase calls for reads, genotypes, or variant calls) with respect to other nucleobase calls, a standard or reference, or for targeted for a particular objective or task. For example, re-engineered sequencing metrics can include modifications to, or combinations of, raw (e.g., unmodified) sequencing metrics. In some embodiments, for instance, the call recalibration system generates one or more of the read-based sequencing metrics, the externally sourced sequencing metrics, and/or the call-model-generated sequencing metrics as re-engineered sequencing metrics. In some cases, re-engineered sequencing metrics refer to sequencing metrics that are generated by the call recalibration system and are therefore proprietary or internal to the call recalibration system and not available to third-party systems. Example re-engineered sequencing metrics include a comparative-mapping-quality-distribution metric indicating a comparison between mapping quality distributions associated with a reference sequence and alternatives supporting nucleotide reads or a comparative-base-quality metric indicating comparisons between base qualities of a reference sequence and alternative supporting nucleotide reads.

As further used herein, the term “genomic coordinate” (or sometimes simply “coordinate”) refers to a particular location or position of a nucleobase within a genome (e.g., an organism's genome or a reference genome). In some cases, a genomic coordinate includes an identifier for a particular chromosome of a genome and an identifier for a position of a nucleobase within the particular chromosome. For instance, a genomic coordinate or coordinates may include a number, name, or other identifier for a chromosome (e.g., chr1 or chrX) and a particular position or positions, such as numbered positions following the identifier for a chromosome (e.g., chr1:1234570 or chr1:1234570-1234870). In some cases, a genomic coordinate refers to a genomic coordinate on a sex chromosome (e.g., chrX or chrY). Consequently, the call recalibration system can determine genotype probabilities and/or variant call classifications for a genotype call (e.g., a variant call) for a genomic coordinate on a sex chromosome. Further, in certain implementations, a genomic coordinate refers to a source of a reference genome (e.g., mt for a mitochondrial DNA reference genome or SARS-CoV-2 for a reference genome for the SARS-CoV-2 virus) and a position of a nucleobase within the source for the reference genome (e.g., mt:16568 or SARS-CoV-2:29001). By contrast, in certain cases, a genomic coordinate refers to a position of a nucleobase within a reference genome without reference to a chromosome or source (e.g., 29727).

Relatedly, as used herein, the term “multiallelic genomic coordinate” refers to a genomic coordinate associated with three or more alleles. For example, a multiallelic genomic coordinate includes a genomic coordinate of a nucleotide sequence where nucleotide reads indicate three or more possible alleles corresponding to the coordinate, such as a reference allele, a first alternate allele, a second alternate allele, and so forth. In some cases, a multiallelic genomic coordinate corresponds to a genomic coordinate where a read pileup occurs or where an insertion occurs. For instance, a multiallelic genomic coordinate can exhibit a multiallelic genotype, such as a 1/2 genotype, where the first allele at the coordinate corresponds to an allele from a first alternate nucleotide sequence and the second allele corresponds to an allele from a second alternate nucleotide sequence.

As indicated above, genomic coordinates within a nucleotide sequence can exhibit different genotypes. For example, a “homozygous reference genotype” refers to a genotype where both nucleotide bases at a given coordinate of a sample nucleotide sequence match a reference nucleotide base of a reference sequence or a reference genome (represented as 0/0). As another example, a “homozygous alternate genotype” refers to a genotype at a given coordinate where both nucleotide bases differ from a reference nucleotide base of a reference sequence or a reference genome (represented as 1/1). As a further example, a “heterozygous genotype” refers to a genotype where the nucleotide bases at a given coordinate are not the same. In some cases, a heterozygous genotype includes a genotype in which one nucleotide base matches a reference nucleotide base and the other nucleotide base differs from the reference nucleotide base (represented as 0/1 or 1/0). For multiallelic genomic coordinates, genotypes can exhibit nucleotide bases from more than one alternate nucleotide base differing from a reference nucleotide base of a reference genome. For instance, a multiallelic heterozygous genotype can be represented as 1/2, where one nucleotide base call matches a first alternate nucleotide base differing from a reference nucleotide base and the other nucleotide base call matches a second alternate nucleotide base differing from the reference nucleotide base.

As noted above, a genomic coordinate includes a position within a reference genome. Such a position may be within a particular reference genome. As used herein, the term “reference genome” refers to a digital nucleic acid sequence assembled as a representative example (or representative examples) of genes and other genetic sequences of an organism. Regardless of the sequence length, in some cases, a reference genome represents an example set of genes or a set of nucleic acid sequences in a digital nucleic acid sequenced determined by scientists as representative of an organism of a particular species. For example, a linear human reference genome may be GRCh38 or other versions of reference genomes from the Genome Reference Consortium. As a further example, a reference genome may include a reference graph genome that includes both a linear reference genome and paths representing nucleic acid sequences from ancestral haplotypes, such as Illumina DRAGEN Graph Reference Genome hg19.

As suggested above, the call recalibration system can utilize a machine learning model to modify sequencing metrics and update a genotype call. As used herein, the term “machine learning model” refers to a computer algorithm or a collection of computer algorithms that automatically improve for a particular task through experience based on use of data. For example, a machine learning model can utilize one or more learning techniques to improve in accuracy and/or effectiveness. Example machine learning models include various types of decision trees, support vector machines, Bayesian networks, or neural networks. In some cases, the call-recalibration-machine-learning model is a series of gradient boosted decision trees (e.g., XGBoost algorithm), while in other cases the call-recalibration-machine-learning model is a random forest model, a multilayer perceptron, a linear regression, a support vector machine, a deep tabular learning architecture, a deep learning transformer (e.g., self-attention-based-tabular transformer), or a logistic regression.

In some cases, the call recalibration system utilizes a call-recalibration-machine-learning model to generate outputs for confirming, modifying, or updating a genotype call based on extracted sequencing metrics. As used herein, the term “call-recalibration-machine-learning model” refers to a machine learning model that generates variant-call classifications. For example, in some cases, the call-recalibration-machine-learning model is trained to generate variant-call classifications indicating various probabilities or predictions for genotype calls (e.g., variant calls) based on the extracted sequencing metrics. Accordingly, in some cases, a call-recalibration-machine-learning model is a variant-call-recalibration-machine-learning model. In some cases, the call-recalibration-machine-learning model is a series of gradient boosted decision trees (e.g., XGBoost algorithm or treelite algorithm for an ensemble of decision trees), while in other cases the call-recalibration-machine-learning model is a random forest model, a multilayer perceptron, a linear regression, a support vector machine, a deep tabular learning architecture, a deep learning transformer (e.g., self-attention-based-tabular transformer), or a logistic regression. In certain embodiments, a call-recalibration-machine-learning model includes multiple sub-models or operates in tandem with another call-recalibration-machine-learning model. For instance, a first call-recalibration-machine-learning model (e.g., an ensemble of gradient boosted trees) generates a first set of variant-call classifications and a second call-recalibration-machine-learning model (e.g., a random forest) generates a second set of variant-call classifications.

Relatedly, the term “variant-call classification” refers to a predicted classification from a call-recalibration-machine-learning model that indicates a probability, score, or other quantitative measurement associated with some aspect of a genotype call based on one or more sequencing metrics extracted from one or more sequencing data files. A variant-call classification can include a specialized prediction depending on the application of a call-recalibration-machine-learning model. In some cases, variant-call classifications for a biallelic genomic coordinate includes (i) a false-positive probability that a genotype call is a false positive, (ii) a genotype-error probability that a genotype for the genotype call is incorrect, and (iii) a true-positive probability that the genotype call is a true positive. As a further example, in embodiments for generating genotype calls for multiallelic genomic coordinates, variant-call classifications can include: (i) a reference probability that a genotype call comprises a homozygous reference genotype at a multiallelic genomic coordinate, (ii) a zygosity-error probability that the genotype call comprises a genotype-zygosity error at a multiallelic genomic coordinate, and (iii) a true-positive variant probability that the genotype call constitutes a true positive variant at a multiallelic genomic coordinate.

In embodiments for generating genotype calls for a haploid genomic coordinate, variant-call classifications can include: (i) a first genotype probability of a first genotype at the genomic coordinate and (ii) a second genotype probability of a second genotype at the genomic coordinate. As suggested above, the first genotype probability can be a probability that a genotype at a genomic coordinate is a haploid reference genotype, and the second genotype probability can be a probability that a genotype at the genomic coordinate is a haploid alternate genotype. In these or other embodiments, such as embodiments for generating genotype calls for genomic coordinates indicated to exhibit homozygous reference genotypes, variant-call classifications can include: (i) a false-positive probability or a homozygous reference classification indicating a probability that a genotype call is a false positive or a homozygous reference genotype, respectively; (ii) a zygosity-error probability or a heterozygous genotype classification indicating a probability that a genotype (e.g., an indication of a heterozygous or homozygous genotype for a variant call at a particular location) is incorrect or a heterozygous genotype, respectively; and/or (iii) a true-positive classification or a homozygous alternate classification indicating a probability that a genotype call is a true positive or a homozygous alternate genotype, respectively. In some cases, the variant-call classifications accordingly represent intermediate scoring metrics and/or a predicted probability that a genotype for a genotype call is accurate.

Accordingly, as further used herein, the term “genotype probability” refers to a likelihood, probability, or score of a particular genotype at a genomic coordinate or genomic region. For instance, a genotype probability includes a likelihood of a homozygous reference genotype, a likelihood of a heterozygous variant genotype, or a likelihood of a homozygous variant genotype at one or more genomic coordinates. In some cases, a genotype probability can refer to a posterior genotype probability. Accordingly, in some cases, a genotype probability determined by a call-recalibration-machine-learning model can be presented in (or modified to be presented in) a posterior genotype probability (GP) field of a VCF or other sequencing data file, such as a recalibrated VCF or other recalibrated sequencing data file. A genotype probability can include a specialized prediction depending on the application of a call-recalibration-machine-learning model, such as for predicting SNPs.

As mentioned, in some embodiments, the call-recalibration-machine-learning model can be a neural network. The term the term “neural network” refers to a machine learning model that can be trained and/or tuned based on inputs to determine classifications or approximate unknown functions. For example, a neural network includes a model of interconnected artificial neurons (e.g., organized in layers) that communicate and learn to approximate complex functions and generate outputs (e.g., generated digital images) based on a plurality of inputs provided to the neural network. In some cases, a neural network refers to an algorithm (or set of algorithms) that implements deep learning techniques to model high-level abstractions in data. For example, a neural network can include a convolutional neural network, a recurrent neural network (e.g., an LSTM), a graph neural network, a self-attention transformer neural network, or a generative adversarial neural network.

As noted above, the call recalibration system can generate variant-call classifications that indicate or reflect a likelihood of identifying a variant at a genomic coordinate. As used herein, the term “variant” refers to a nucleobase or multiple nucleobases that do not align with, differs from, or varies from a corresponding nucleobase (or nucleobases) in a reference sequence or a reference genome. For example, a variant includes a SNP, an indel, or a structural variant that indicates nucleobases in a sample nucleotide sequence that differ from nucleobases in corresponding genomic coordinates of a reference sequence. Along these lines, a “variant nucleotide base call” (or simply “variant call”) refers to a nucleotide base call comprising a variant at a particular genomic coordinate. Conversely, a “non-variant nucleotide base call” (or simply “non-variant call”) refers to a nucleotide base call comprising a non-variant at a genomic coordinate.

As mentioned, in one or more embodiments, the call recalibration system extracts or determines sequencing metrics from one or more existing sequencing data files. As used herein, the term “sequencing data file” refers to a digital file that includes genetic sequencing information concerning genotype calls or nucleotide reads generated by one or more genomic sequencing procedures. Such sequencing information may include, for example, nucleotide reads, alignment and mapping information, nucleotide reads at one or more genomic coordinates, and so forth. In some embodiments, the call recalibration system accesses multiple sequencing data files to extract or determine sequencing metrics, such as an alignment data file and a genotype-call data file. In one or more embodiments, the call recalibration system accesses a sequencing data file that includes all of the aforementioned sequencing information consolidated into a single file.

As mentioned, in some embodiments, the call recalibration system modifies data fields corresponding to a genotype-call data file, such as a variant call file. As used herein, the term “genotype-call data file” refers to a digital file that indicates or represents one or more genotype calls (e.g., including reference and/or variant calls) compared to a reference genome along with other information pertaining to the genotype calls (e.g., variant calls). For example, a genotype-call data file can include a variant call file, such as but not limited to a variant call format (VCF) file (as well as a genomic variant call format (gVCF) file). Alternatively, as a further example, genotype-call data file can include a General Feature Format (GFF), a Genome Variant Format (GVF), or other suitable data file comprising genotype calls for a sample nucleotide sequence.

As further used herein, a “variant call file” refers to a particular genotype-call data file that comprises a text file format that contains information about variants at specific genomic coordinates. For instance, a variant call file can include meta-information lines, a header line, and data lines where each data line contains information about a single genotype call (e.g., a single variant). As described further below, the call recalibration system can generate different versions of genotype-call data files, including a pre-filter variant call file comprising variant genotype calls that either pass or fail a quality filter for base-call-quality metrics or a post-filter variant call file comprising variant genotype calls that pass the quality filter but excludes variant genotype calls that fail the quality filter.

As also mentioned, in one or more embodiments, the one or more sequencing data files from which the call recalibration system extracts or determines sequencing metrics include an alignment data file containing information from a read processing and mapping procedure. As used herein, the term “alignment data file” refers to a digital file that indicates mapping and alignment information for nucleotide reads of a sample nucleotide sequence. For example, an alignment data file can include a binary alignment map (BAM) file, a compressed reference-oriented alignment map (CRAM) file, or another file indicating nucleotide reads of a sample nucleotide sequence.

In some embodiments, the call recalibration system modifies data fields corresponding to metrics of a genotype call associated with a variant call file, such as fields for call quality, genotype, and genotype quality. As used herein, the term “call quality” when used with respect to a data field in a variant call file refers to a measure or an indication of a likelihood or a probability that a variant exists at a given location. Accordingly, a call quality field (or QUAL field) corresponding to a VCF file may include a base-call-quality metric, such as a PHRED-scaled quality or Q score, representing a probability that a genomic coordinate of a sample genome includes a variant. Similarly, a “genotype quality” when used with respect to a field refers to a likelihood or a probability that a particular predicted genotype for a nucleobase call is correct.

As noted, in some embodiments, the call recalibration system extracts or determines sequencing metrics from one or more sequencing data files, such as a genotype-call data file containing genotype calls output by a call generation model. As used herein, the term “call generation model” refers to a probabilistic model that generates sequencing data from nucleotide reads of a sample nucleotide sequence, including nucleobase calls, variant calls, and/or genotype calls along with associated metrics. Accordingly, in some cases, a call generation model may be a variant call generation model. For example, in some cases, a call generation model refers to a Bayesian probability model that generates variant calls based on nucleotide reads of a sample nucleotide sequence. Such a model can process or analyze sequencing metrics corresponding to read pileups (e.g., multiple nucleotide reads corresponding to a single genomic coordinate), including mapping quality, base quality, and various hypotheses including foreign reads, missing reads, joint detection, and more. A call generation model may likewise include multiple components, including, but not limited to, different software applications or components for mapping and aligning, sorting, duplicate marking, computing read pileup depths, and variant calling. In some cases, a call generation model refers to an ILLUMINA DRAGEN model for variant calling functions and mapping and alignment functions (e.g., a DRAGEN variant caller or “DRAGEN VC”).

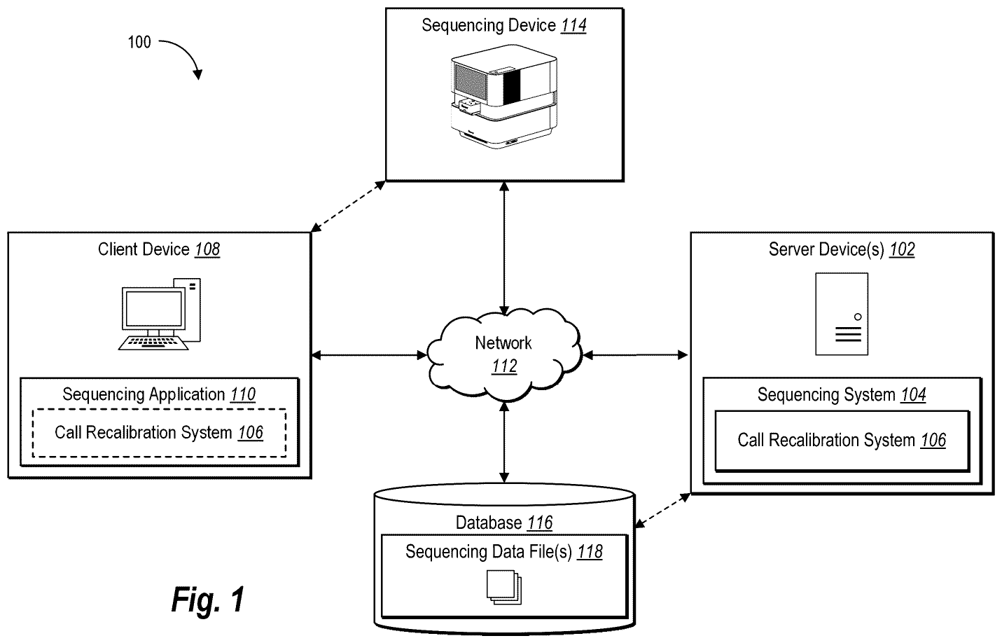

The following paragraphs describe the call recalibration system with respect to illustrative figures that portray example embodiments and implementations. For example, FIG. 1 illustrates a schematic diagram of a system environment (or “environment”) 100 in which a call recalibration system 106 operates in accordance with one or more embodiments. As illustrated, the environment 100 includes one or more server device(s) 102 connected to a client device 108 and a sequencing device 114 via a network 112. While FIG. 1 shows an embodiment of the call recalibration system 106, this disclosure describes alternative embodiments and configurations below.

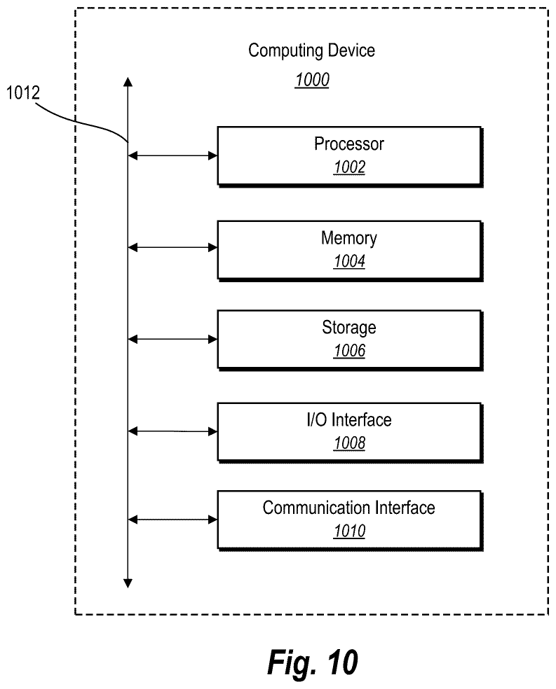

As shown in FIG. 1, the server device(s) 102, the client device 108, and the sequencing device 114 can communicate with each other via the network 112. The network 112 comprises any suitable network over which computing devices can communicate. Example networks are discussed in additional detail below with respect to FIG. 10.

As indicated by FIG. 1, the sequencing device 114 comprises a device for sequencing a nucleic acid polymer. In some embodiments, the sequencing device 114 analyzes nucleic acid segments or oligonucleotides extracted from genomic samples to generate nucleotide reads or other data utilizing computer implemented methods and systems (described herein) either directly or indirectly on the sequencing device 114. More particularly, the sequencing device 114 receives and analyzes, within nucleotide-sample slides (e.g., flow cells), nucleic acid sequences extracted from genomic samples. In one or more embodiments, the sequencing device 114 utilizes SBS to sequence nucleic acid polymers into nucleotide reads. In addition or in the alternative to communicating across the network 112, in some embodiments, the sequencing device 114 bypasses the network 112 and communicates directly with the client device 108.

As further indicated by FIG. 1, the server device(s) 102 may generate, receive, analyze, store, and transmit digital data, such as data for determining nucleotide base calls or sequencing nucleic acid polymers. As shown in FIG. 1, the sequencing device 114 may send (and the server device(s) 102 may receive) call data from the sequencing device 114. The server device(s) 102 may also communicate with the client device 108. In particular, the server device(s) 102 can send data to the client device 108, including sequencing data files, such as genotype-call data files or alignment data files, or other information indicating nucleotide base calls, sequencing metrics, error data, or other metrics associated with a nucleotide base call or genotype calls.

In some embodiments, the server device(s) 102 comprise a distributed collection of servers where the server device(s) 102 include a number of server devices distributed across the network 112 and located in the same or different physical locations. Further, the server device(s) 102 can comprise a content server, an application server, a communication server, a web-hosting server, or another type of server. In some cases, the server device(s) 102 are located at a same physical location as the sequencing device 114.

As further shown in FIG. 1, the server device(s) 102 can include a sequencing system 104. Generally, the sequencing system 104 analyzes call data, such as sequencing metrics received from the sequencing device 114, to determine nucleotide base sequences for nucleic acid polymers. For example, the sequencing system 104 can receive raw data from the sequencing device 114 and can determine a nucleotide base sequence for a sample nucleotide sequence (e.g., genomic sample). In some embodiments, the sequencing system 104 determines the sequences of nucleotide bases in DNA and/or RNA segments or oligonucleotides. In addition to processing and determining sequences for nucleic acid polymers, the sequencing system 104 also generates a genotype-call data file, such as a variant call file, indicating one or more genotype calls and/or variant calls for one or more genomic coordinates.

As mentioned, and as illustrated in FIG. 1, the call recalibration system 106 analyzes call data, such as sequencing metrics from the sequencing device 114 stored in existing sequencing data files, to recalibrate genotype calls for sample nucleotide sequences that were previously generated (e.g., by a call generation model). The call recalibration system 106 includes a call-recalibration-machine-learning model. In some embodiments, the call recalibration system 106 determines sequencing metrics for sample nucleotide sequences based on information stored in existing sequencing data files. Based on data derived or prepared from the sequencing metrics, the call recalibration system 106 trains and applies a call-recalibration-machine-learning model to recalibrate genotype calls for the sample sequence corresponding to genomic coordinates. The call recalibration system 106 further utilizes the call-recalibration-machine-learning model to generate sets of variant-call classifications to update or modify the genotype calls (e.g., variant calls). Based on such data, for example, the call recalibration system 106 can update data fields corresponding to genotype-call data file, such as a variant call file, to update a genotype call (e.g., a variant call) for improved accuracy. In some embodiments, the call recalibration system 106 outputs an updated variant call file (or other format of genotype-call data file) with the modified or updated genotype calls and/or variant calls.

As further illustrated and indicated in FIG. 1, the client device 108 can generate, store, receive, and send digital data. In particular, the client device 108 can receive sequencing metrics from the sequencing device 114. Furthermore, the client device 108 may communicate with the server device(s) 102 to receive a genotype-call data file, such as a variant call file, comprising genotype calls and/or other metrics, such as a call-quality, a genotype indication, and a genotype quality. The client device 108 can accordingly present or display information pertaining to the genotype call within a graphical user interface to a user associated with the client device 108.

The client device 108 illustrated in FIG. 1 may comprise various types of client devices. For example, in some embodiments, the client device 108 includes non-mobile devices, such as desktop computers or servers, or other types of client devices. In yet other embodiments, the client device 108 includes mobile devices, such as laptops, tablets, mobile telephones, or smartphones. Additional details regarding the client device 108 are discussed below with respect to FIG. 10.

As further illustrated in FIG. 1, the client device 108 includes a sequencing application 110. The sequencing application 110 may be a web application or a native application stored and executed on the client device 108 (e.g., a mobile application, desktop application). The sequencing application 110 can include instructions that (when executed) cause the client device 108 to receive data from the call recalibration system 106 and present, for display at the client device 108, data from a variant call file and/or an updated variant call file. Furthermore, the sequencing application 110 can instruct the client device 108 to display a visualization of sequencing metrics of a nucleotide base call or genotype call.

As further illustrated in FIG. 1, the call recalibration system 106 may be located on the client device 108 as part of the sequencing application 110 or on the sequencing device 114. Accordingly, in some embodiments, the call recalibration system 106 is implemented by (e.g., located entirely or in part) on the client device 108. In yet other embodiments, the call recalibration system 106 is implemented by one or more other components of the environment 100, such as the sequencing device 114. In particular, the call recalibration system 106 can be implemented in a variety of different ways across the server device(s) 102, the network 112, the client device 108, and the sequencing device 114. For example, the call recalibration system 106 can be downloaded from the server device(s) 102 to the client device 108 and/or to the sequencing device 114 where all or part of the functionality of the call recalibration system 106 is performed at each respective device within the environment 100.

As further illustrated in FIG. 1, the environment 100 includes a database 116. The database 116 can store information, such as sequencing data file(s) 118, sample nucleotide sequences, nucleotide reads, nucleotide base calls, genotype calls (e.g., variant calls), and sequencing metrics. In some embodiments, the server device(s) 102, the client device 108, and/or the sequencing device 114 communicate with the database 116 (e.g., via the network 112) to store and/or access information, such as the sequencing data file(s) 118, sample nucleotide sequences, nucleotide reads, nucleotide base calls, genotype calls (e.g., variant calls), and sequencing metrics. In some cases, the database 116 also stores one or more models, such as a call-recalibration-machine-learning model.

Though FIG. 1 illustrates the components of environment 100 communicating via the network 112, in certain implementations, the components of environment 100 can also communicate directly with each other, bypassing the network 112. For instance, and as previously mentioned, in some implementations, the client device 108 communicates directly with the sequencing device 114. Additionally, in some embodiments, the client device 108 communicates directly with the call recalibration system 106. Moreover, the call recalibration system 106 can access one or more databases housed on or accessed by the server device(s) 102 or elsewhere in the environment 100.

As mentioned above, the call recalibration system 106 can generate modified or updated genotype calls based on information extracted or determined from one or more existing sequencing data files. As also mentioned, in some implementations, the call recalibration system 106 exhibits improvements to efficiency and/or accuracy over existing sequencing systems in generating recalibrated and/or improved genotype calls for previously analyzed sample nucleotide sequences.

To further illustrate, FIG. 2A shows an overview of an existing sequencing system generating updated sequencing data for a previously analyzed sample nucleotide sequencing system. Specifically, FIG. 2A shows two existing call generation models, a call generation model 204a and an updated call generation model 204b, generating genotype calls from base call data 202 and outputting the respective genotype calls to respective sequencing data file(s) 210a and updated sequencing data file(s) 210b.

As shown in the illustrated example, existing methods for updating genotype calls and other sequencing information can include utilizing an updated call generation model (e.g., a newer call generation model that exhibits improved results), such as the updated call generation model 204b, to re-analyze data from a sequencing device, such as the base call data 202. As suggested above, in some cases, the updated call generation model 204b includes an updated version of a machine-learning-based variant caller a machine-learning-based variant caller that the call generation model 204a lacks. In some cases, numerous sequencing data files (e.g., sequencing data files comprising nucleotide base reads for thousands of sample nucleotide sequences) are updated in this manner, requiring extensive computational resources to re-process the base call data for each of the previously analyzed genomic samples.

For example, as illustrated, analysis of the base call data 202 by the call generation model 204a includes a procedure for read processing and mapping 206a and a procedure for genotype calling 208a. When newly analyzed by the updated call generation model 204b, the renewed analysis generally requires performance of an updated read processing and mapping procedure 206b and an updated genotype calling procedure 208b.

In contrast, as shown in FIG. 2B, the call recalibration system 106 confirms, updates, or modifies genotype calls based on information extracted or determined from the existing sequencing data file(s) 210a that were previously generated by the call generation model 204a. In other words, the call recalibration system 106 generates recalibrated sequencing data file(s) 210c without directly reprocessing the base call data 202. Instead, as shown, the call recalibration system 106 utilizes a call-recalibration-machine-learning model 212 to generate the recalibrated sequencing data file(s) 210c based on sequencing metrics extracted or determined from the existing data file(s) 210a. Accordingly, the call recalibration system 106 takes advantage of previous analysis of the base call data 202 by the call generation model 204a, including the read processing and mapping 206a and genotype calling 208a, by utilizing the trained call-recalibration-machine-learning model 212 to update the sequencing data within the existing sequencing data file(s) 210a. As illustrated by FIG. 7 and other figures, the call recalibration system 106 saves significant computer processing runtime and expands the flexibility of the type of processor for variant calling by generally following the procedure in FIG. 2B rather than the procedure in FIG. 2A.

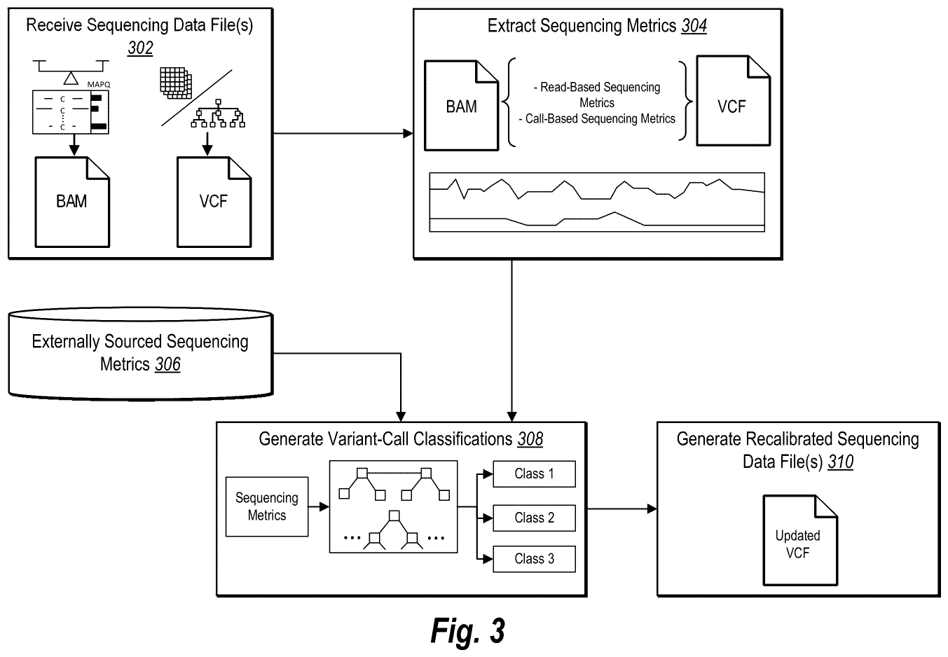

As indicated above, the call recalibration system 106 can generate variant-call classifications based on sequencing metrics extracted or determined from one or more existing sequencing data files. In particular, the call recalibration system 106 can determine variant-call classifications from extracted sequencing metrics utilizing a call-recalibration-machine-learning model and can determine or update various metrics associated with a genotype call from the generated variant-call classifications. FIG. 3 illustrates an example overview of the call recalibration system 106 determining variant-call classifications based on extracted sequencing metrics in accordance with one or more embodiments.

As illustrated in FIG. 3, the call recalibration system 106 performs an act 302 to receive one or more existing sequencing data file(s). In particular, in some embodiments, the call recalibration system 106 receives a first sequencing data file, such as an alignment data file (e.g., a BAM file or a CRAM file), comprising data for nucleotide reads. In addition, in some embodiments, the call recalibration system 106 receives a second sequencing data file, such as a genotype-call data file (e.g., a VCF file or a gVCF file) having one or more genotype calls at one or more genomic coordinates. Alternatively, in one or more embodiments, the call recalibration system 106 can receive the aforementioned sequencing data in one or more sequencing data files of an alternative format, such as, but not limited to, a single sequencing data file having data for nucleotide reads and genotype calls. Moreover, in some embodiments, the call recalibration system 106 receives one or more sequencing data files having additional sequencing information, as further discussed below in relation to subsequent figures.

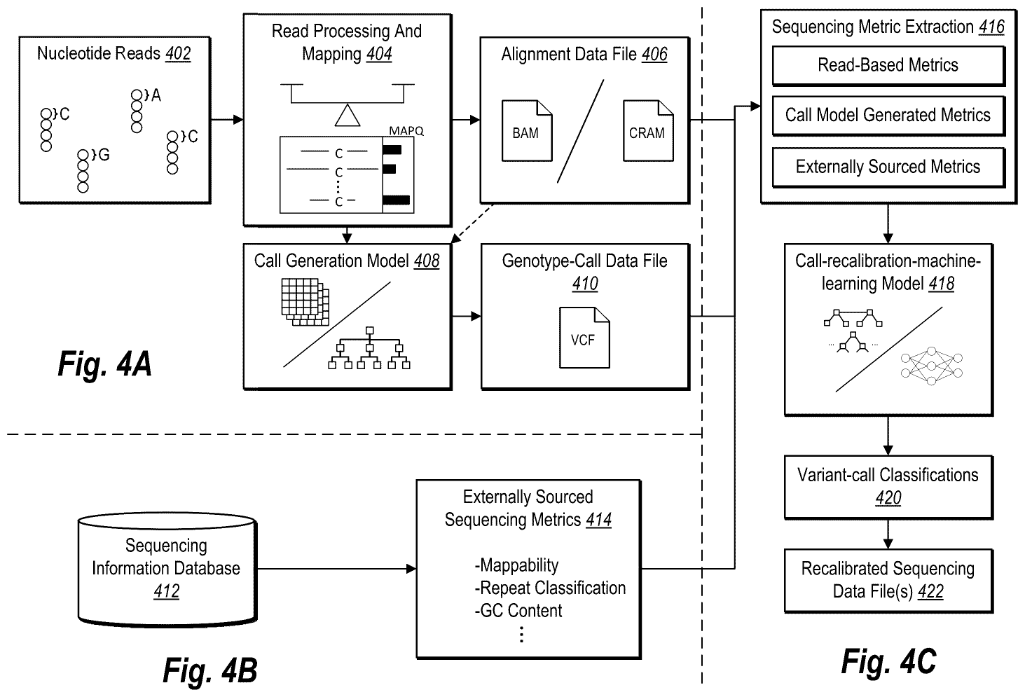

As also illustrated in FIG. 3, the call recalibration system 106 performs an act 304 to extract sequencing metrics from the one or more sequencing data files received during act 302. In particular, the call recalibration system 106 extracts or determines sequencing metrics, such as read-based sequencing metrics and call-based sequencing metrics. For example, the call recalibration system 106 extracts or determines sequencing metrics that indicate various attributes or data in relation to various genotype calls from a sample nucleotide sequence (e.g., a genomic sample). In some embodiments, the call recalibration system 106 determines or extracts different sequencing metrics for generating genotype calls associated with different variant types, such as SNPs and indels. Moreover, in some embodiments and as shown in FIG. 3, the call recalibration system 106 performs and act 306 to access or receive externally sourced sequencing metrics. Additional detail regarding determining the various types of sequencing metrics is provided below with reference to FIGS. 4A-4C.

As further illustrated in FIG. 3, the call recalibration system 106 performs an act 308 to generate variant-call classifications. More specifically, the call recalibration system 106 generates (or updates or refines) variant-call classifications from extracted sequencing metrics utilizing a call-recalibration-machine-learning model. To elaborate, in some embodiments, the call recalibration system 106 utilizes the call-recalibration-machine-learning model to process or analyze one or more extracted sequencing metrics and to generate a set of classifications (e.g., predicted probabilities associated with genotypes). For instance, the call recalibration system 106 generates, utilizing the call-recalibration-machine-learning model, a set of variant-call classifications (represented in FIG. 3 as “Class 1,” “Class 2,” and “Class 3”) that indicate certain probabilities associated with a genotype of a corresponding genotype call based on the sequencing metrics.

In some embodiments, the call recalibration system 106 generates different variant-call classifications for different applications and/or for different genomic coordinates. By using different version of a call-recalibration-machine-learning model, for example, the call recalibration system 106 generates a first set of variant-call classifications for multiallelic genomic coordinates, generates a second set of variant-call classifications for haploid genomic coordinates, and generates a third set of variant-call classifications for genomic coordinates indicated to exhibit homozygous reference genotypes. In certain embodiments, the call recalibration system 106 generates the same variant-call classifications for different applications and/or for different genomic coordinates but utilizes them differently or utilizes different information associated with the variant-call classifications. Additional detail regarding generating variant-call classifications is provided below with reference to subsequent figures.

As further illustrated in FIG. 3, the call recalibration system 106 also performs an act 310 to generate one or more recalibrated sequencing data files. For example, the call recalibration system 106 determines a modified or updated genotype call (e.g., a variant call) based on the variant-call classifications and indicates any such modifications in the recalibrated sequencing data file(s) (e.g., in an updated VCF file). More particularly, the call recalibration system 106 modifies or updates a genotype call for a sample nucleotide sequence at a genomic coordinate within a reference genome. To determine the updated or modified genotype call, in some embodiments, the call recalibration system 106 edits or updates certain existing genotype calls (i.e., from the sequencing data file(s) received at act 302) based on the variant-call classifications generated by the call-recalibration-machine-learning model.

To elaborate, the call recalibration system 106 extracts or determines sequencing metrics (e.g., one or more of the same sequencing metrics used to generate the variant-call classifications) to analyze a genotype call from the extracted sequencing metrics. For example, the call recalibration system 106 applies a number of Bayesian probabilistic models or algorithms to derive various probabilities for different nucleotide bases, quality metrics, mapping metrics, joint metrics, and other data occurring within the sample nucleotide sequence. From the probabilistic models, the call recalibration system 106 determines an updated genotype call (e.g., a call indicating a difference or sameness to a reference base from a reference genome) that indicates a pair of predicted nucleotide bases for a genomic sample at a corresponding genomic coordinate.

As further illustrated in FIG. 3, in certain implementations, the call recalibration system 106 utilizes the variant-call classifications (e.g., as determined via the act 308) to generate, recalibrate, modify, or augment the existing genotype call. To elaborate, the call recalibration system 106 utilizes probabilities associated with the variant-call classifications to determine or update certain metrics associated with a genotype call. For example, the call recalibration system 106 modifies data fields corresponding to a sequencing data file (e.g., a genotype-call data file, such as a variant call file), for metrics, such as call quality, genotype, and genotype quality (or others as described below).

In some cases, the call recalibration system 106 extrapolates from the variant-call classifications to determine metrics corresponding to an existing sequencing data file, such as call quality, genotype, and genotype quality associated with the genotype call. For instance, by utilizing a zygosity-error probability, the call recalibration system 106 can remedy certain errors in or associated with an existing genotype call. Indeed, if the call recalibration system 106 determines a high false-positive probability for a genotype call, then the call recalibration system 106 applies the call-recalibration-machine-learning model to function as a variant filter to modify (e.g., reduce) a call quality associated with the genotype call. As another example, the call recalibration system 106 utilizes a zygosity-error probability to modify a genotype and/or a genotype quality of a genotype call in cases where systems would previously filter out or doubly penalize heterozygous/homozygous (het/hom) errors (e.g., where the system generates a genotype call that is incorrect which further results in missing a genotype call that is correct).

Relatedly, in some embodiments, the call recalibration system 106 produces an updated genotype call at a biallelic (or other type of) genomic coordinate by determining to change an existing genotype call from homozygous to heterozygous, from heterozygous to homozygous, from a variant call to a reference call, from a reference call to a variant call, or any combination of the foregoing. To illustrate, in some implementations, the call recalibration system 106 changes a heterozygous-variant genotype call or a homozygous-variant genotype call reported in the one or more sequencing data files to a homozygous-reference genotype call at a genomic coordinate. In another implementation, the call recalibration system 106 changes a homozygous-reference genotype call or a homozygous-variant genotype call reported in the one or more sequencing data files to a heterozygous-variant genotype call at the genomic coordinate. In yet another implementation, the call recalibration system 106 changes a heterozygous-variant genotype call or a homozygous-reference genotype call reported in the one or more sequencing data files to a homozygous-variant genotype call at the genomic coordinate.

In certain embodiments, the call recalibration system 106 considers a single variant-call classification to modify a data field for a genotype call (e.g., a call quality, a genotype, or a genotype quality). By contrast, in some embodiments, the call recalibration system 106 considers multiple variant-call classifications at once (e.g., in a weighted combination) to modify or update one or more data fields for call quality, genotype, and/or genotype quality. Additional detail regarding generating and modifying genotype calls is provided below with reference to subsequent figures.

As mentioned above, in some implementations, the call recalibration system 106 is not necessarily limited to modifying or updating a single genotype call included in the one or more sequencing data files. For instance, in one or more embodiments, the call recalibration system 106, having extracted sequencing metrics for a genotype call, generated variant-call classifications for the genotype call, and/or modified the genotype call based on the variant-call classifications, extracts sequencing metrics for a particular genotype call from the one or more sequencing data files. By utilizing the call-recalibration-machine-learning model, the call recalibration system 106 generates one or more variant-call classifications indicating an accuracy of the particular genotype call indicated by the one or more sequencing data files. Based on the one or more additional variant-call classifications, the call recalibration system 106, in some implementations, modifies or updates a base-call-quality metric (e.g., Q score), genotype-probability metric, genotype-likelihood metric, or genotype-quality metric for the particular genotype call. For example, in some implementations, the call recalibration system 106 determines a modified base-call-quality metric, modified genotype-probability metric, modified genotype-likelihood metric, or modified genotype-quality metric for the particular genotype call that falls below a base-call-quality threshold or other corresponding threshold. Based on the modified base-call-quality metric not satisfying the base-call-quality threshold, the call recalibration system 106 annotates the particular genotype call, within a recalibrated sequencing data file, to indicate that the modified base-call-quality metric falls below the base-call-quality threshold. Similarly, based on the modified genotype-probability metric, modified genotype-likelihood metric, or modified genotype-quality metric not satisfying a genotype-probability threshold, a genotype-likelihood threshold, or a genotype-quality threshold, respectively, the call recalibration system 106 annotates the particular genotype call, within a recalibrated sequencing data file, to indicate that the modified genotype-probability metric, modified genotype-likelihood metric, or modified genotype-quality metric falls below the corresponding threshold.

As mentioned above, in certain described embodiments, the call recalibration system 106 extracts sequencing metrics for nucleotide base calls or genotype calls at particular genomic coordinates. In particular, the call recalibration system 106 extracts sequencing metrics such as read-based sequencing metrics, externally sourced sequencing metrics, and call-model-generated sequencing metrics from one or more sequencing data files for calls corresponding to existing nucleotide reads from a sample nucleotide sequence. FIGS. 4A-4C illustrate extracting sequencing metrics in accordance with one or more embodiments. Specifically, FIG. 4A illustrates receiving sequencing data files (an alignment data file 406 and a genotype-call data file 410) having sequencing data for nucleotide reads 402 of a sample nucleotide sequence, FIG. 4B illustrates determining externally sourced sequencing metrics 414, and FIG. 4C illustrates extracting sequencing metrics 416 and generating recalibrated sequencing data file(s) 422.

As illustrated in FIG. 4A, the call recalibration system 106 accesses, retrieves, or otherwise obtains nucleotide reads 402. In particular, in some embodiments, the nucleotide reads 402 are previously determined utilizing the sequencing device 114 comprising nucleotide base calls for regions from a sample nucleotide sequence (e.g., sample genome). For example, the nucleotide reads 402 can be generated utilizing sequencing-by-synthesis (SBS) techniques and/or Sanger sequencing techniques to determine nucleotide base calls for oligonucleotide clusters from wells in a flow cell and/or via fluorescent tagging. More specifically, the nucleotide reads 402 are generated utilizing cluster generation and SBS chemistry to sequence millions or billions of clusters in a flow cell. During SBS chemistry, for each cluster, the call nucleotide base calls from the nucleotide reads 402 are stored and, in some embodiments, provided directly to the call recalibration system 106, for every cycle of sequencing via real-time analysis (RTA) software.

As further illustrated in FIG. 4A, in some embodiments, the alignment data file 406, such as a BAM file or a CRAM file, is generated by read processing and mapping 404. For example, the read processing and mapping 404 includes utilizing real-time analysis (RTA) software to store base call data in the form of individual base call data files (or BCLs). In some cases, the read processing and mapping 404 further includes converting the BCL files into sequence data (e.g., via BCL to FASTQ conversion) to be analyzed by a call generation model 408 to determine genotype calls for the nucleotide reads 402.

In particular, in certain embodiments, the read processing and mapping 404 includes aligning nucleotide reads with a reference genome or receiving information pertaining to the read alignment. Specifically, the read processing and mapping 404 determines which nucleotide base(s) of a given read align with which genomic coordinate of a reference sequence (or receives information indicating alignment). Different reads have different lengths and include different nucleotide bases. Accordingly, in some cases, the read processing and mapping 404 includes analysis of each nucleotide of each read to determine (or receives information indicating) where the read “fits” in relation to a reference sequence—e.g., where the bases within the read align with bases in the reference. In some cases, the read processing and mapping 404 includes alignment of many reads at a single genomic coordinate, thus resulting in a read pileup.

In certain embodiments, the call recalibration system 106 performs additional statistical tests to determine or detect differences between metrics associated with a reference nucleotide sequence and metrics associated with alternative supporting nucleotide reads. Through these statistical tests, the call recalibration system 106 re-engineers raw sequencing metrics to determine read-based sequencing metrics. In some cases, the call recalibration system 106 extracts raw sequencing metrics that include one or more of (i) alignment metrics for quantifying alignment of sample nucleotide sequences with genomic coordinates of an example nucleotide sequence (e.g., a reference genome or a nucleotide sequence from an ancestral haplotype), (ii) depth metrics for quantifying depth of nucleotide base calls for sample nucleotide sequences at genomic coordinates of the example nucleotide sequence, or (iii) call-quality metrics for quantifying quality of nucleotide base calls (e.g., genotype calls) for sample nucleotide sequences at genomic coordinates of the example nucleotide sequence. For instance, the call recalibration system 106 extracts mapping-quality metrics (e.g., the MAPQ metrics indicated in FIG. 4A), soft-clipping metrics, or other alignment metrics that measure an alignment of sample sequences with a reference genome. As another example, the call recalibration system 106 extracts forward-reverse-depth metrics (or other such depth metrics) or callability metrics for variant genotype calls (or other such call-quality metrics).

As just mentioned, in some embodiments, the call recalibration system 106 re-engineers the raw sequencing metrics extracted from the alignment data file 406 (or other sequencing data files) to generate read-based sequencing metrics that are more informative for comparing metrics associated with a reference nucleotide sequence with metrics associated with various supporting alternative nucleotide reads. For example, the call recalibration system 106 extracts various metrics for a sample sequence in relation to a reference sequence and further extracts various metrics for the sample sequence in relation to alternative supporting sequences. In addition, in some embodiments, the call recalibration system 106 performs comparative analyses between metrics associated with the reference sequence and the metrics associated with the alternative supporting reads.

For instance, the call recalibration system 106 compares how nucleotide bases of a sample nucleotide sequence (e.g., sample genome) map to a reference sequence with how the nucleotide bases map to various alternative supporting reads. In some cases, the call recalibration system 106 determines mapping qualities associated with the reference sequence to compare with mapping qualities associated with alternative supporting reads. For example, the call recalibration system 106 determines mapping quality statistics reflecting differences in the distribution of reads supporting a reference sequence versus reads supporting alternative alleles.

In these or other cases, the call recalibration system 106 determines mismatch counts between the sample sequence and the reference sequence and between the reference sequence and alternative supporting reads. The call recalibration system 106 further compares the mismatch counts to extract a comparative-mismatch-count metric. Further, the call recalibration system 106 determines soft-clipping metrics for the sample sequence in relation to the reference sequence and further extracts soft-clipping metrics in relation to alternative supporting reads. The call recalibration system 106 also compares the soft clipping metrics between the reference sequence and the alternative supporting reads to generate a comparative-soft-clipping metric. Further still, the call recalibration system 106 compares base-call-quality metrics in relation to the reference sequence and alternative supporting reads and/or compares query positions of the sample sequence in relation to the reference sequence with those in relation to alternative supporting reads.

As further illustrated in FIGS. 4A and 4C, the call recalibration system 106 utilizes the comparisons and/or other statistical tests to extract the read-based sequencing metrics from information within the alignment data file 406 (or other sequencing data files), including: i) a comparative-mapping-quality-distribution metric indicating a mapping quality distribution comparing mapping qualities in relation to the reference sequence and mapping qualities in relation to alternative supporting reads, ii) a comparative-secondary-mapping-alignment metric indicating a comparison between secondary mapping in relation to bases in the reference sequence and bases in alternative supporting reads, iii) a comparative-mismatch-count metric indicating a comparison between mismatched nucleotide bases in relation to the reference sequence and mismatched bases in relation to alternative supporting reads, iv) a comparative-soft-clipping metric indicating a comparison between soft-clipping metrics in relation to the reference sequence and soft-clipping metrics in relation to alternative supporting reads, v) one or more comparative-read-depth metrics indicating comparisons between read depths of the nucleotide reads 402 and one or more average read depths (e.g., local average read depths at a particular genomic coordinate and global average read depths across a number genomic coordinates in a region), vi) one or more comparative-base-quality metric indicating comparisons between base qualities in relation to the reference sequence and base qualities in relation to alternative supporting reads (e.g., for overall base quality, early base quality, and late base quality in the nucleotide reads 402), vii) a comparative-query-position metric indicating a comparison between query positions in relation to the reference sequence and query positions in relation to alternative supporting reads, viii) one or more contextual-information metrics indicating homopolymers and periodicity of nucleotide base calls, ix) a strand-bias metric indicating a strand bias associated with one or more of the nucleotide reads 402, and x) a read-direction-bias metric indicating a read direction bias associated with the nucleotide reads 402. In some cases, the call recalibration system 106 extracts or re-engineers additional or alternative read-based sequencing metrics from information stored in the alignment data file 406 (or other sequencing data files).

In addition to the read-based sequencing metrics, as illustrated in FIGS. 4A and 4C, the call recalibration system 106 extracts the call-model-generated sequencing metrics from the genotype-call data file 410 generated by the call generation model 408. For example, the call generation model 408 determines sequence data based on the read processing and mapping 404. In some cases, the call generation model 408 generates the sequence data as part of one or more digital files, such as BCL and FASTQ files.

To generate such files, in some embodiments, the sequencing device 114 (or the call generation model 408) utilizes cluster generation and SBS chemistry to sequence millions or billions of clusters in a flow cell. During SBS chemistry, for each cluster, the sequencing device 114 (or the call generation model 408) stores nucleotide base calls from the nucleotide reads 402 for every cycle of sequencing via real-time analysis (RTA) software. The sequencing device 114 (or the call generation model 408) utilizes RTA software to further store base call data in the form of individual base call data files (or BCLs). In some cases, the sequencing device 114 (or the call generation model 408) further converts the BCL files into sequence data (e.g., via BCL to FASTQ conversion). For instance, the sequencing device 114 (or the call generation model 408) generates a FASTQ file from the nucleotide reads 402, where the FASTQ file includes sequence data.

In some cases, the call generation model 408 generates the sequence data for each cluster that passes an initial quality filter from a sample sequence. For example, the call generation model 408 generates entries for each cluster, where each entry includes four lines (or four items of sequence data): i) a sequence identifier with information about the sequencing run and the cluster, ii) nucleotide base calls that make up the sequence (e.g., a sequence of A, C, T, G, and/or N calls), iii) a separator (e.g., a “+” sign), and iv) base-call-quality metrics indicating probabilities of correctness for the nucleotide base calls (Phred+33 encoded).

As further illustrated in FIG. 4A, the call generation model 408 processes or analyzes the sequence data to generate genotype calls. Indeed, in some embodiments, the call recalibration system 106 extracts the call-model-generated sequencing metrics by re-engineering raw sequencing metrics (e.g., raw sequencing metrics within the sequence data utilized by the call generation model 408 and stored within one or more sequencing data files, such as BCL or FASTQ files). In particular, the call generation model 408 includes mapping-and-alignment components to map and align nucleotide base calls from the sequence data. In addition, the call generation model 408 includes variant calling components to generate genotype calls (e.g., reference-base calls such as variant calls or non-variant calls) from the sequence data and stores the genotype calls within the genotype-call data file 410 (e.g., a VCF or gVCF file). In some cases, the call recalibration system 106 extracts the call-model-generated sequencing metrics that have previously been generated utilizing the mapping-and-alignment components and the variant calling components of the call generation model 408 by accessing the genotype-call data file 410.

To illustrate examples of the call-model-generated sequencing metrics, in some cases, the call recalibration system 106 extracts (variant calling metrics including one or more of: i) a base-call-quality metric (e.g., DRAGEN QUAL score) indicating a quality score for genotype calls generated via the call generation model 408, ii) a call model generated-foreign-read-detection metric (e.g., foreign read detection (FRD) score) indicating a probability that one or more of the nucleotide reads 402 in a pileup might be foreign reads (e.g., their true location is elsewhere in the reference sequence), iii) a call model generated-base-quality-dropoff metric (e.g., base quality dropoff (BQD) score) indicating a probability of base quality dropoff based on one or more of strand bias, error position in a thread, or low mean base quality over a subset of the nucleotide reads 402, iv) average read depths, v) indel statistics (e.g., a polymerase chain reaction or “PCR” curve) and/or vi) hidden Markov model (HMM) statistics (or reconstructs hidden Markov model (HMM) statistics utilizing Concise Idiosyncratic Gapped Alignment Report (CIGAR)), vii) a secondary-alignment metric indicating a probability that a secondary genotype call is correct, viii) a base-context metric indicating contextual information for nucleotide around a genotype call, iv) a nearby-call metric indicating nearby (e.g., adjacent or within a threshold degree of separation from) a genotype call, x) a joint-detection metric indicating a probability of detecting a joint corresponding to two or more overlapping nucleotide base calls, xii) read-filtering metrics indicating threshold quality metrics or other metrics for filtering out nucleotide base calls with low mapping quality, base quality, or other quality metrics, or others. In some cases, the call recalibration system 106 extracts the call-model-generated sequencing metrics from internal (e.g., proprietary, and model-specific) variables that reflect interacting processing paths, corner cases, and difficult predictions/decisions.

While not listed in FIG. 4A or 4C, the call-model-generated sequencing metrics include, but are not limited to, variant calling metrics extracted via the variant calling components of the call generation model 408 and stored within (or otherwise determined from) an existing version of the genotype-call data file 410. In addition or in the alternative to the examples of the call-model-generated sequencing metrics described above, in some cases, the call recalibration system 106 extracts or generates (e.g., via metric re-engineering) variant calling metrics including one or more of: i) a number of samples in a population, ii) a number of reads processed for generating genotype calls, a number of variants (e.g., SNPs, indels, and MNPs), iii) a number of biallelic sites (e.g., genomic coordinates that contain two observed alleles), iv) a number of multiallelic sites (e.g., a number of sites in a variant call file that contain three or more observed alleles), v) a number of SNPs, vi) numbers of different types of indels (e.g., homozygous insertions, heterozygous insertions, and heterozygous deletions), vii) a total number of heterozygous indels (e.g., insertion+deletion, insertion+SNP, or deletion+SNP), viii) a number of de novo SNPs (e.g., SNPs with de novo quality metrics that satisfy a threshold level), ix) a number of de novo indels (e.g., indels with de novo quality metrics that satisfy a threshold level), x) a number of de novo MNPs (e.g., MNPs with de novo quality metrics that satisfy a threshold level, xi) a number of SNPs in a first chromosome divided by a number of SNPs in a second chromosome, xii) a number of SNP transitions, xiii) a number of SNP transversions, xiv) a number of heterozygous variants, xv) a number of homozygous variants, xvi) a ratio between the number of heterozygous variants and the number of homozygous variants, xvii) a number of variants detected within a dbSNP reference file, and/or xviii) a total number of variants minus the number detected within the dbSNP file.

Additionally, the call-model-generated sequencing metrics can include mapping-and-alignment sequencing metrics extracted via the mapping-and-alignment components of the call generation model 408 and stored within an existing version of the genotype-call data file 410 and/or the alignment data file 406. For instance, the call recalibration system 106 extracts or generates (e.g., via metric re-engineering) mapping-and-alignment metrics including one or more of: i) a number of total input reads, ii) a number of duplicate marked reads, iii) a number of duplicate marked and mate reads removed, iv) a number of unique reads, v) a number of reads with mate sequenced, vi) a number of reads without mate sequenced, vii) indications of reads that fail quality checks, viii) indications of mapped reads, ix) a number of unique and mapped reads, x) a number of unmapped reads, xi) a number of singleton reads (e.g., where the read is mapped but the paired mate could not be read), xii) a number of paired reads, xiii) a number of properly paired reads (e.g., where both reads in a pair are mapped and fall within an acceptable range from each other based on an estimated insert length distribution), xiv) a number of discordant reads (e.g., not properly paired reads), xv) a number of paired reads mapped to different chromosomes, xvi) a number of paired reads mapped to different chromosomes that also have a mapping-quality metric of 10 or greater, xvii) percentages of reads within indels R1 and R2, xviii) percentages of bases in R1 and R2 that are soft clipped, xix) a number of mismatched bases in R1 and R2, xx) a number of bases with a base quality of at least 30 (e.g., total and/or in R1 or R2), xxi) a number of alignments (e.g., total alignments, secondary alignments, and/or supplementary alignments), xxii) an estimated read length, and xxiii) an estimated sample contamination.

Turning now to FIG. 4B, in some cases, the call recalibration system 106 generates, extracts, or determines externally sourced sequencing metrics 414. In particular, the call recalibration system 106 determines externally sourced sequencing metrics 414 from one or more databases external to the call recalibration system 106, such as a sequencing information database 412 (e.g., the database 116). For example, the call recalibration system 106 accesses sequencing metrics that are generic or applicable to sequencing nucleotides generally. In addition, the call recalibration system 106 accesses or determines sequencing information about a particular reference sequence (e.g., stored within the sequencing information database 412). In some cases, the call recalibration system 106 determines externally sourced sequencing metrics 414 including: i) a mappability metric indicating an ease or difficult of mapping a particular nucleotide sequence (or a particular nucleotide read or nucleotide base call), ii) a guanine-cytosine-content metric indicating a count (or a dropout or a mean) of guanine-cytosine content in a reference nucleotide sequence (e.g., reference genome), iii) a replication-timing metric indicating a time required to replicate a particular number of nucleotides from a reference sequence, iv) one or more DNA-structure-metrics indicating DNA structures of a reference sequence (e.g., reference genome), v) a conservation metric indicating a measure of sequence conservation across multiple species (e.g., a measure of change relative to an average), and/or others.

As discussed above (e.g., in relation to FIG. 3) and as illustrated in FIG. 4C, the call recalibration system 106 extracts sequencing metrics 416 from sequencing data files previously generated by various sequencing and base-calling processes, such as read processing and mapping 404 and base-calling by call generation model 408 (see FIG. 4A). In particular, the call recalibration system 106 utilizes a call-recalibration-machine-learning model 418 to generate variant-call classifications 420 and, based on the variant-call classifications 420, generates as output a recalibrated sequencing data file(s) 422.

In one or more embodiments, the call recalibration system 106 extracts (or reconstructs), from one or more sequencing data files, additional or alternative sequencing metrics, including read-based sequencing metrics, call-model-generated sequencing metrics, and/or externally sourced sequencing metrics. For example, the call recalibration system 106 extracts the sequencing metrics in following table, where each of the metrics belongs to one or more of the read-based sequencing metrics, call-model-generated sequencing metrics, and/or externally sourced sequencing metrics.

| Sequencing Metric | Description |
|---|---|
| Mappability | Lookup files of mappability scores by genomic |
| | position |
| Variant type | SNP or indel |
| Length | + for insertion, − for deletion, 0 for SNP |
| Indel_class_ref | For multiallelic positions, allows +/− |
| Indel_class_alt | For multiallelic positions, allows +/− |
| Ref_softclip | Number of softclips in reference-supporting |
| | reads |
| Alt_softclip | Number of softclips in alternate-supporting reads |
| Querypos_p | Statistical test of query position difference |
| | between reference-supporting reads and |
| | alternate-supporting reads |
| Leftpos_p | Statistical test of leftmost read position |
| | difference between reference-supporting reads |
| | and alternate-supporting reads |
| Seqpos_p | Statistical test of sequencing position difference |
| | between reference-supporting reads and |
| | alternate-supporting reads |
| Mapq_p | Statistical test of mapping quality difference |
| | between reference-supporting reads and |
| | alternate-supporting reads |
| Baseq_p | Statistical test of base quality difference between |
| | reference-supporting reads and alternate- |
| | supporting reads |
| Ref_baseq | Base quality of reference-supporting reads |
| Alt_baseq | Base quality of alternate-supporting reads |
| Context | Integer field capturing a five-base context |
| | around variant position |
| Major_mismatches_mean | Mean number of mismatches in reference- |
| | supporting reads |
| Minor_mismatches_mean | Mean number of mismatches in alternate- |
| | supporting reads |
| Mismatches_p | Statistical test of mismatch difference between |
| | reference-supporting reads and alternate- |
| | supporting reads |
| AF | Alternate allele frequency |
| AF_other | Allele frequency for any other allele |
| Dp | Sequence depth at position |
| AF_without_mapq0 | Alternate allele frequency after removing mapq0 |
| | reads (e.g., reads where MAPQ = 0) |
| Dp_without_mapq0 | Depth at position after removing mapq0 reads |
| Mapq_p_without_mapq0 | Statistical test of mapping quality difference |
| | between reference-supporting reads and |
| | alternate-supporting reads after removing mapq0 |
| | reads |
| Mosaic_likelihood | Calculated likelihood of a genetic mosaicism |
| Het_likelihood | Calculated likelihood of a heterozygous |
| | genotype |
| Refhom likelihood | Calculated likelihood of a homozygous reference |
| | genotype |
| Althom_likelihood | Calculated likelihood of a homozygous alternate |
| | genotype |
| Mapq_difference | Difference in average MAPQ between reference- |
| | supporting reads and alternate-supporting reads |
| Mapq_ref | Average MAPQ on reference-supporting reads |
| Mapq_alt | Average MAPQ on alternate-supporting reads |
| Mapq_difference_without_mapq0 | Difference in average MAPQ between reference- |
| | supporting reads and alternate-supporting reads |
| | after removing mapq0 reads |
| Mapq_ref_without_mapq0 | Average MAPQ on reference-supporting reads |
| | after removing mapq0 reads |
| Mapq_alt_without_mapq0 | Average MAPQ on alternate-supporting reads |
| | after removing mapq0 reads |
| Encoded_position | Normalized position along chromosome |
| Qual | VC quality score |
| Gt | VC-derived genotype |
| Gq | VC-derived genotype quality score |
| Gerp200, gerp1000, gerp10000 | Gerp scores in window around variant position |
| Gc20, gc50, gc100, gc250, gc500, gc1000, | GC bias in window around variant position |
| gc2500, gc5000, gc10000, gc25000, gc75000 | |
| LowMap | Low mappability region flag |
| Homopolymer_100 | Window around known homopolymers in |
| | reference genome |
| Replication_timing | Score for replication timing at variant position |
| Ref_base | Encoded reference base |
| Alt_base | Encoded alternate base |
| Transition | Flag for a transition variant |
| FS | Fisher strand bias metric |
| ReadPosRankSum | Evidence of bias in position of alleles within |
| | reads that support them, between reference and |
| | alternate alleles |
| SOR | Strand bias |
| Max_depth | Maximum depth in active region |
| Avg_depth | Average depth in active region |
| Repeat_period | Repeat period at variant position |
| Repeat_length | Repeat length at variant position |
| Ins_gop | Insert gap opening penalty estimated at variant |
| | position |
| Del_gop | Delete gap opening penalty estimated at variant |
| | position |
| Is_columnwise_event | Flags variants that come from a simplified |
| | columnwise caller instead of full HMM-based |
| | calling |
| Base counts in window around variant position | Number of base counts within a window around |
| | a variant position |
| Ratio of soft clipping | Ref_softclip / alt_softclip |
| Insert size | Size of insertion |
| Population statistics | Population genotyping statistics |
| Dinuc flag | Flags dinucleotides, such as CPGs at variant |
| | position |
| Rosetta scores | Measure of repeatability of calls at locations or |
| | variant positions |
| RepeatMasker class | Indication of class within RepeatMasker |
| | database |
| g-quad flag | Indicates presence of g-quadruplex |
| Larger context window | Integer field capturing a context size (e.g., ten or |
| | twenty bases) around a variant position |

As mentioned above, in certain described embodiments, the call recalibration system 106 generates sets of machine learning predictions for different variant types using the sequencing metrics described above. In particular, in some embodiments, the call recalibration system 106 utilizes a call-recalibration-machine-learning model to generate genotype probabilities (for SNPs) or another call-recalibration-machine-learning model to generate variant-call classifications (for indels) corresponding to various genomic coordinates. In addition, the call recalibration system 106 updates of modifies a genotype call by generating an updated genotype-call data file, such as variant call file (e.g., a recalibrated variant call file) based on the genotype probabilities and/or the variant-call classifications.

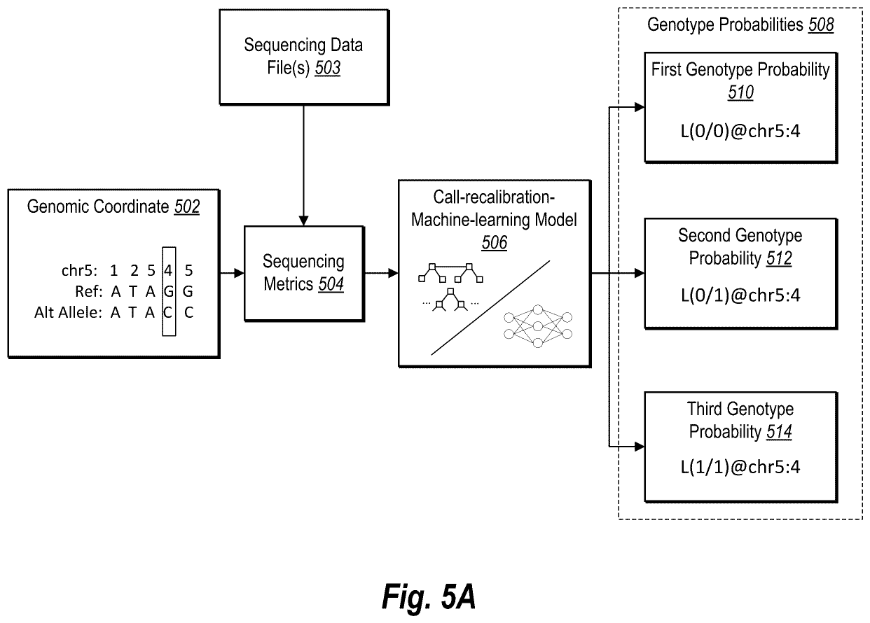

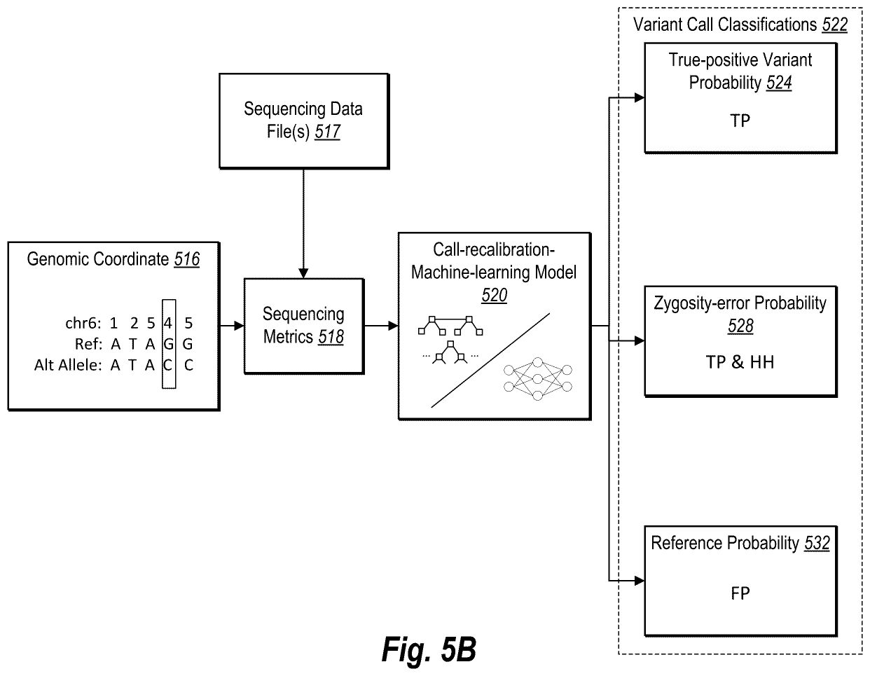

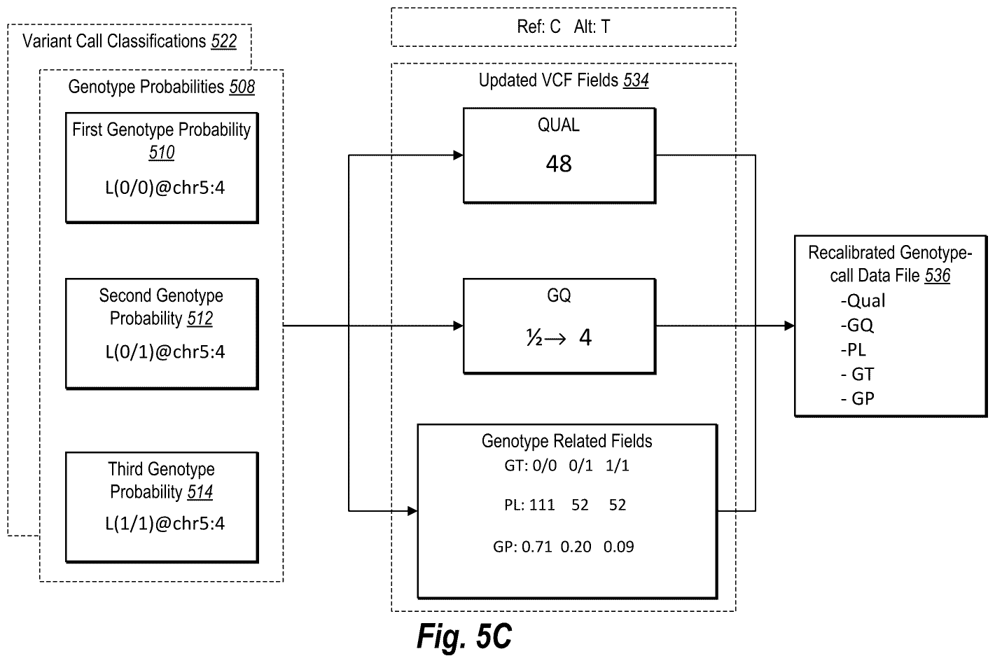

In accordance with one or more embodiments, FIGS. 5A-5C illustrate the call recalibration system 106 generating one or both of genotype probabilities and variant-call classifications, generating a genotype call based on such likelihoods and/or classifications, and generating a recalibrated or updated variant call file comprising the genotype call based on such probabilities and/or classifications. For example, FIG. 5A illustrates the call recalibration system 106 using a call-recalibration-machine-learning model to generate genotype probabilities for (biallelic) SNPs based on sequencing metrics corresponding to existing genotype calls in accordance with one or more embodiments. FIG. 5B illustrates the call recalibration system 106 using a call-recalibration-machine-learning model to generate variant-call classifications for indels (or multiallelic SNPs or variant types other than biallelic SNPs) based on sequencing metrics corresponding to existing genotype calls in accordance with one or more embodiments. Thereafter, FIG. 5C illustrates the call recalibration system 106 generating an updated variant call file comprising recalibrated genotype calls based on the genotype probabilities and/or the variant-call classifications in accordance with one or more embodiments.

As illustrated in FIG. 5A, the call recalibration system 106 identifies a genomic coordinate 502. For instance, the call recalibration system 106 identifies the genomic coordinate 502 from nucleobase calls corresponding to a sample nucleotide sequence or based on haplotype data corresponding to the genomic coordinate 502. In some cases, the call recalibration system 106 identifies the genomic coordinate 502 by determining (i) one or more nucleobase calls from nucleotide reads covering a genomic coordinate and (ii) that the one or more nucleobase calls satisfy one or more threshold sequencing metrics (e.g., a base-call-quality metric of Q30). Additionally or alternatively, in certain embodiments, the call recalibration system 106 identifies the genomic coordinate 502 from a database comprising a haplotype reference panel correlated with specific genomic coordinates. Regardless of the identification method, in some cases, the call recalibration system 106 uses information from one or more sequencing data file(s) 503, such as information previously generated and stored by a call generation model (e.g., a variant caller as part of a call generation model), to identify the genomic coordinate 502.

In addition, the sequencing data file(s) 503 accessed by the call recalibration system 106 also include an existing genotype call (e.g., a genotype call generated and stored by a call generation model in a variant call file). To elaborate, the sequencing data file(s) 503 include information determined by a call generation model (e.g., a DRAGEN VC Caller) to generate the existing genotype call to predict presence (or absence) of a variant (or a particular genotype) at the genomic coordinate 502. As described, the call generation model generates the existing genotype call by analyzing or processing sequencing metrics 504 (or a subset of the sequencing metrics 504, such as read-based sequencing metrics and externally sourced sequencing metrics), which are also available to the call recalibration system 106 from the sequencing data file(s) 503. In addition, the call generation model generates and stores (e.g., in a genotype-call data file) some of the sequencing metrics 504 (e.g., the call-model-generated sequencing metrics) as part of predicting the existing genotype call.

Indeed, the call recalibration system 106 extracts (or reconstructs), from one or more existing sequencing data files, sequencing metrics 504 for the genomic coordinate 502. In particular, the call recalibration system 106 extracts sequencing metrics associated with nucleotide reads, generated by the call generation model, or retrieved from another external source, as described above. Based on the sequencing metrics 504, the call recalibration system 106 further generates genotype probabilities 508 that together can indicate a measure of confidence or a probability that the genomic coordinate 502 includes or exhibits a SNP. The genotype probabilities 508 represent an example of variant-call classifications.

Specifically, as shown in FIG. 5A, the call recalibration system 106 utilizes a call-recalibration-machine-learning model 506 to generate the genotype probabilities 508. For example, the call-recalibration-machine-learning model 506 analyzes or processes the extracted sequencing metrics 504 and the existing genotype call as inputs to generate, as outputs, the genotype probabilities 508, including: (i) a first genotype probability 510 that the existing genotype call is a homozygous reference genotype at the genomic coordinate 502 (e.g., “L(0/0)@chr5:4”), (ii) a second genotype probability 512 that the existing genotype call is a heterozygous variant genotype at the genomic coordinate 502 (e.g., “L(0/1)@chr5:4”), and (iii) a third genotype probability 514 that the existing genotype call is a homozygous variant genotype at the genomic coordinate 502 (e.g., “L(1/1)@chr5:4”).

As mentioned, the call recalibration system 106 generates the genotype probabilities 508 to predict whether an SNP occurs at the genomic coordinate 502. To predict whether an indel occurs at a genomic coordinate, in some embodiments, the call recalibration system 106 generates a different set of machine learning predictions. Specifically, the call recalibration system 106 generates variant-call classifications that indicate presence (or absence) of an indel (or a multiallelic SNP or another variant type other than a biallelic SNP) at a genomic coordinate of a sample sequence.

As shown in FIG. 5B, the call recalibration system 106 utilizes a call-recalibration-machine-learning model 520 to generate variant-call classifications 522. To elaborate, the call recalibration system 106 utilizes call-recalibration-machine-learning model 520 to generate the variant-call classifications 522 based on sequencing metrics 518 extracted (or reconstructed) from sequencing data file(s) 517 and an existing genotype call associated with a genomic coordinate 516. For instance, in some embodiments, the call recalibration system 106 identifies multiallelic genomic coordinates, such as the genomic coordinate 516, and feeds the call-recalibration-machine-learning model 520 sequencing metrics for the multiallelic genomic coordinates. Indeed, similar to the discussion above regarding generating genotype probabilities for a biallelic SNP, the call recalibration system 106 likewise extracts (or reconstructs) sequencing metrics 518 associated with the genomic coordinate 516, including read-based sequencing metrics, call-model-generated sequencing metrics, and externally sourced sequencing metrics. For instance, the call recalibration system 106 analyzes a subset of the sequencing metrics 518 (e.g., read-based sequencing metrics and/or externally sourced sequencing metrics) extracted from the sequencing data file(s) 517 for determining the existing genotype call (e.g., indicating a particular genotype or variant at the genomic coordinate 516). In some cases, when generating the sequencing data file(s) 517, a call generation model generates the subset of the sequencing metrics 518 (e.g., call-model-generated sequencing metrics) associated with the genomic coordinate 516.

In generating the variant-call classifications 522 for the genomic coordinate 516, the call recalibration system 106 utilizes the call-recalibration-machine-learning model 520. Particularly, the call recalibration system 106 utilizes the call-recalibration-machine-learning model 520 to generate: (i) a true-positive variant probability 524 that the existing genotype call (e.g., from an initial VCF file of the sequencing data file(s) 517) is a true positive variant call at the genomic coordinate 516, (ii) a zygosity-error probability 528 that the existing genotype call (e.g., from an initial VCF file of the sequencing data file(s) 517) comprises a genotype-zygosity error at the genomic coordinate 516, and (iii) a reference probability 532 that the existing genotype call at the genomic coordinate 516 is a homozygous reference genotype (or a false positive). In some cases, the variant-call classifications 522 are mutually exclusive.

As further shown in FIG. 5B, the true-positive variant probability 524 is represented by “TP.” The symbol “TP” represents the probability that an input (x) is a true positive variant in an existing genotype-call data file (e.g., an initial VCF file of the sequencing data file(s) 517) of the sequencing data file(s) 517, where “TP” can be formulated as P(tp|x)). By contrast, the zygosity-error probability 528 is represented by “HH.” The symbol “TP&HH” represents the probability that the input (x) is not a true positive and is a het-hom error in the existing genotype-call data file (e.g., an initial VCF file of the sequencing data file(s) 517). Further, the reference probability 532 is represented by “FP,” which indicates the probability that the input (x) is a false positive and can be formulated as P(fp|x)).

To elaborate on the zygosity-error probability 528, the call recalibration system 106 determines probabilities that predicted genotypes (e.g., existing genotype calls) at the genomic coordinate 516 are incorrect genotypes (e.g., a genotype incorrectly identified by the call generation model) or include an incorrect allele. In some cases, the call recalibration system 106 determines, based on the sequencing metrics 518, a probability that a zygosity error (e.g., a het/hom error) exists at the genomic coordinate 516—e.g., where the alternate base is correct but the genotype is wrong—or a probability that the nucleobase calls represent either the wrong genotype altogether or the wrong allele(s) in the existing genotype call. For example, when determining a probability that a zygosity error exists, the call recalibration system 106 determines a probability that an alternate base call represented as “1” is correct, but the genotype is incorrect, such as a probability of incorrectly determining a 0/1 genotype call (e.g., A/T) instead of a correct 1/1 genotype call (e.g., T/T) (or vice versa when the correct genotype call is 0/1).

By determining the zygosity-error probability 528, the call recalibration system 106 can fix inaccuracies of existing sequencing systems where incorrect calls are often indels. In particular, the call recalibration system 106 can more accurately generate genotype calls for genomic coordinates corresponding to indels where existing sequencing systems would determine a genotype call represent an incorrect genotype that represents an incorrect allele resulting from a long inserted or deleted sequence.

As further illustrated in FIG. 5B, the call recalibration system 106 utilizes the call-recalibration-machine-learning model 520 to generate the true-positive variant probability 524. In particular, the call recalibration system 106 generates the true-positive variant probability 524 based on the sequencing metrics 518 for an existing genotype call at the genomic coordinate 516. In some cases, a true-positive variant probability indicates a probability of a correct variant call genotype at the genomic coordinate 516. For example, the call recalibration system 106 generates a probability that the existing genotype call for the genomic coordinate 516 is correct as determined by the call generation model and stored in the sequencing data file(s) 517.

Continuing to FIG. 5C, in some embodiments, the call recalibration system 106 utilizes the genotype probabilities 508 and/or the variant-call classifications 522 to update one or more data fields or variant call file fields (“VCF” fields) associated with a variant call file (e.g., of the sequencing data files accessed by the call recalibration system 106). For example, the call recalibration system 106 generates a recalibrated genotype-call data file 536 (in this case, a recalibrated variant call file) based on the genotype probabilities 508. In some cases, the call recalibration system 106 generates a single recalibrated variant call file that combines data (e.g., updated genotype calls and/or updated sequencing metrics) from the genotype probabilities 508 for SNPs and from the variant-call classifications 522 for indels.

As shown, the call recalibration system 106 generates updated VCF fields 534 that indicate, or correspond to, updated sequencing metrics for an existing genotype call. Specifically, the call recalibration system 106 generates one set of the updated VCF fields 534 based on the genotype probabilities 508 for a set of genomic coordinates. Further, the call recalibration system 106 generates another set of the updated VCF fields 534 based on the variant-call classifications 522 for a different set of genomic coordinates. In some cases, the call recalibration system 106 modifies or updates only certain VCF fields and does not update others based on the genotype probabilities 508 and/or the variant-call classifications 522.

In other cases, the call recalibration system 106 does not update VCF fields. When updating or modifying genotype calls, for instance, the call recalibration system 106 does not update certain fields, such as a genotype (GT) field, based on the genotype probabilities 508 and/or the variant-call classifications 522. Indeed, in some cases, the call recalibration system 106 does not modify or update a GT field because there may not be enough information to determine a new or updated genotype at a genomic coordinate.

To illustrate one embodiment, FIG. 5C depicts the call recalibration system 106 generating the updated VCF fields 534 for a genotype (GT) of 1/2, where cytosine represents a reference base (shown as “Ref: C”) at a genomic coordinate for an allele corresponding to the reference genome, thymine represents a first alternate base (“Alt: T”) at the genomic coordinate for a different allele. But FIG. 5C merely depicts examples of a possible reference base and possible alternate bases at a genomic coordinate. The call recalibration system 106 can generate genotype probabilities 508 and variant-call classifications 522 to modify corresponding sequencing metrics in VCF fields for various other reference bases and alternate bases at genomic coordinates.

As further illustrated in FIG. 5C, the call recalibration system 106 generates an updated base-call-quality metric in a base-call-quality (QUAL) field. More specifically, the call recalibration system 106 modifies or updates a base-call-quality metric based on the genotype probabilities 508 and/or the variant-call classifications 522 to indicate an accuracy of a genotype call. As shown, the updated base-call-quality field indicates a QUAL score of 48 for a variant at the corresponding genomic coordinate. In this example, the updated base-call-quality metric (e.g., QUAL score of 48) represents a score for any type of variant at the corresponding genomic coordinate. In addition, the call recalibration system 106 generates a modified or updated genotype quality (GQ) field. For instance, based on the variant-call classifications 522, the call recalibration system 106 generates a modified or updated genotype quality metric indicating a likelihood or a probability that a predicted genotype at a genomic coordinate is correct. As shown, for instance, the updated genotype quality field indicates a genotype quality metric for a genotype call with a heterozygous genotype (e.g., a GQ score of 4 for a genotype of 1/2) for a multiallelic coordinate.

In one or more embodiments, the call recalibration system 106 further generates or updates genotype probability fields and (in some cases) uses the genotype probability fields to rank alleles. To elaborate, the call recalibration system 106 generates an updated GT field by ordering candidate genotype calls at a genomic coordinate according to respective probabilities of belonging at the genomic coordinate 502. For example, the call recalibration system 106 determines probabilities associated with a plurality of genotypes where each diploid genotype is composed of a pair of alleles. As another example, the call recalibration system 106 determines relative probabilities associated with a plurality of alleles (e.g., from a reference genome, a first alternate allele, and a second alternate allele) of belonging at a multiallelic genomic coordinate.

In some embodiments, the call recalibration system 106 also (or alternatively) generates metrics for a PHRED-scale Likelihood (PL) field as part of the updated VCF fields. For example, the call recalibration system 106 generates metrics for a PL field that can indicate genotypes, such as homozygous reference, heterozygous, and homozygous alternate genotypes (e.g., with PL field nomenclature 9/0/3, respectively).

In one or more embodiments, the call recalibration system 106 generates allele-specific probabilities or likelihoods based on a relative probability of a genotype call corresponding to an allele from a call generation model versus any other (non-reference) genotype identified by a call-recalibration-machine-learning model. For instance, in some embodiments, the call recalibration system 106 indicates relative probability scores for each allele corresponding to respective genotype calls in PL fields indicating normalized PHRED-scale likelihoods for genotypes and/or Genotype probability (GL) fields indicating log-scaled likelihoods (e.g., log 10-scaled) of data (e.g., sequencing metrics) given a called genotype.

As motivation for modifying certain VCF fields for an SNP, in some cases, the call recalibration system 106 utilizes a call-recalibration-machine-learning model to generate the genotype probabilities 508 (whose probabilities sum to 1). In particular, the call-recalibration-machine-learning model may generate the first genotype probability 510 as 0.1, the second genotype probability 512 as 0.2, and the third genotype probability 514 as 0.7. Based on the genotype probabilities 508 in such an example, the call recalibration system 106 generates the updated genotype probability fields by updating GT fields, GP fields, and PL fields using a combination of information from the call-recalibration-machine-learning model and the sequencing data file(s) previously generated by a call generation model.

As further illustrated in FIG. 5C, the call recalibration system 106 updates PL fields for different genotypes (GT). According to the normalized scale of a PL score, a relatively lower score (e.g., PL 0) for a genotype represents a relatively higher likelihood of the genotype being present at a genomic coordinate; and a relatively higher score (e.g., PL 101) for the genotype represents a relatively lower likelihood of the genotype being present at the genomic coordinate. For example, the call recalibration system 106 determines a PL score of 111 for the 0/0 genotype, a PL score of 52 for the 0/1 genotype, and a PL score of 52 for the 1/1 genotype. Accordingly, in FIG. 5C, the PL score of 52 indicates genotypes with the highest likelihood or the selected genotype (e.g., the 0/1 and the 1/1 genotypes) and the PL score of 111 represents the lowest likelihood (e.g., a 0/0 genotype).

In some cases, the call recalibration system 106 generates the updated genotype probability fields as a ranking of a plurality of alleles identified via the call generation model (without utilizing a call-recalibration-machine-learning model). In other cases, the call recalibration system 106 utilizes a specialized version of a call-recalibration-machine-learning model that is trained to generate the updated genotype probabilities fields based on the genotype probabilities 508 and/or the variant-call classifications 522.

As further illustrated in FIG. 5C, the call recalibration system 106 generates or updates a genotype-call data file, such as an initial variant call file, to create the recalibrated genotype-call data file 536. For example, the call recalibration system 106 generates the recalibrated genotype-call data file 536 from the updated VCF fields 534 corresponding to the genotype probabilities 508 and the variant-call classifications 522, respectively. Thus, the call recalibration system 106 generates the recalibrated genotype-call data file 536 for an SNP genotype call based on the genotype probabilities 508. As indicated above, in some embodiments, the call recalibration system 106 generates a recalibrated genotype-call data file that merges data for SNPs and indels from both the genotype probabilities 508 and the variant-call classifications 522.

The call recalibration system 106 can generate the recalibrated genotype-call data file 536 to include the updated VCF fields 534, including a base-call-quality metric, a genotype quality metric, and/or updated genotype probability fields. For instance, the call recalibration system 106 selects VCF fields from existing genotype calls generated by a call generation model to include within a recalibrated genotype-call data file. In some embodiments, however, the call recalibration system 106 does not select fields but instead generates new VCF fields for a recalibrated genotype-call data file by using a call-recalibration-machine-learning model to process the genotype probabilities 508 and the variant-call classifications 522.

As mentioned, in some cases, the call recalibration system 106 updates only certain fields while other fields, such as a genotype (GT) field, remain unchanged. For instance, the call recalibration system 106 updates the genotype quality field and the based call quality field. For other data fields such as normalized PHRED-scale likelihoods (PL) for genotypes and posterior genotype probability (GP), the call recalibration system 106 either: (i) maintains the field as-is, (ii) removes the field, or (iii) updates fields to reflect GQ for the called genotype and Class 0 output 0/0. In some cases, the call recalibration system 106 maintains the relative probabilities of other genotypes with respect to the called genotype to ensure consistent updates and that the called genotype is highest. In certain embodiments, by updating only the values for 0/0 and 1/2, the call recalibration system 106 maintains distances of other genotypes from the called genotype. By updating only certain fields, the call recalibration system can more efficiently generate (recalibrated and/or merged) genotype-call data files, without regenerating entirely new genotype-call data files (as done by some prior systems) and/or updating every field (even those that are unchanged by new predictions).

Within (or as a result of generating) a recalibrated genotype-call data file, the call recalibration system 106 can include or update one or more output genotype calls (e.g., variant calls) associated with a genomic coordinate, as determined based on the updated VCF fields 534. Indeed, to generate an updated genotype call, the call recalibration system 106 can predict nucleobases from candidate alleles at the genomic coordinate (e.g., according to their respective probabilities and metrics indicated by the recalibrated variant call file). Thus, the call recalibration system 106 can generate the recalibrated genotype-call data file 536 comprising updated genotype calls for particular genomic coordinates or confirmed genotype calls for particular genomic coordinates. Similarly, the call recalibration system 106 can generate the recalibrated genotype-call data file 536 comprising one or more of a modified base-call-quality metric, a modified genotype-probability metric, a modified genotype metric, a modified genotype-likelihood metric, or a modified genotype-quality metric for the confirmed or modified genotype call.

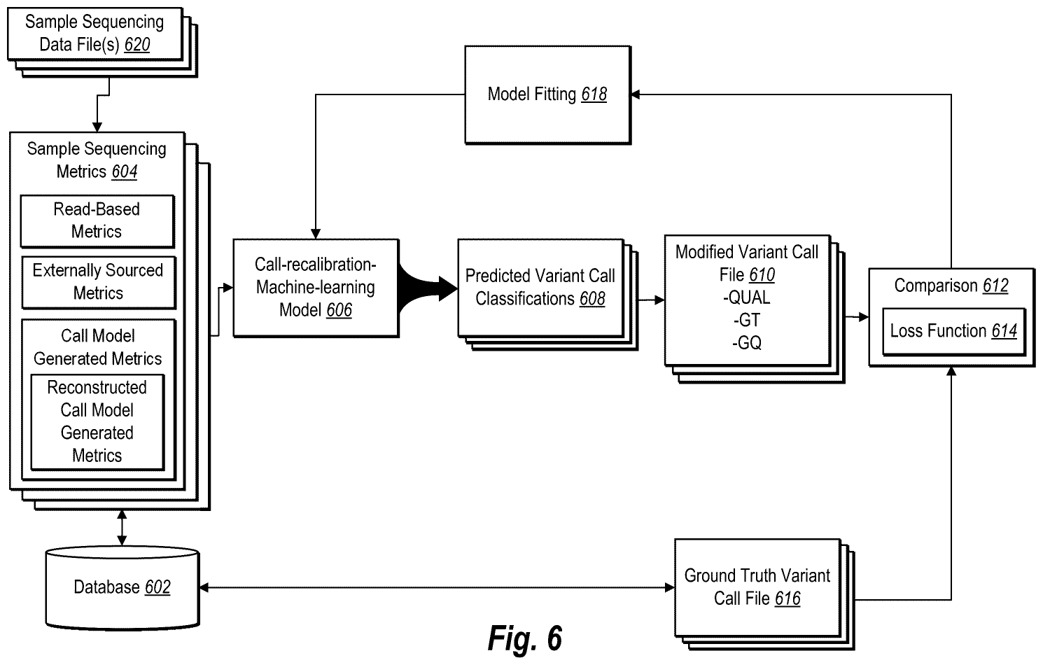

As mentioned above, in certain embodiments, the call recalibration system 106 trains or tunes a call-recalibration-machine-learning model (e.g., the call-recalibration-machine-learning model 418, 506, or 520). In particular, the call recalibration system 106 utilizes an iterative training process to fit a call-recalibration-machine-learning model by adjusting or adding decision trees or learning parameters that result in accurate variant-call classifications (e.g., variant-call classifications 420, genotype probabilities 508, or variant-call classifications 522). For example, FIG. 6 illustrates training a call-recalibration-machine-learning model in accordance with one or more embodiments.

As illustrated in FIG. 6, the call recalibration system 106 accesses sample sequencing data file(s) 620 (e.g., sequencing data file(s) generated utilizing an existing call generation model, such as call generation model 204a) and extracts (or reconstructs) sample sequencing metrics 604 from the sample sequencing data file(s) 620 and receives or obtains some metrics (e.g., externally sourced metrics) from a database 602 (e.g., the database 116). For example, the call recalibration system 106 extracts (or reconstructs) sample sequencing metrics including sample read-based metrics, sample externally sourced sequencing metrics, and sample call-model-generated sequencing metrics.

As mentioned, in some embodiments, the call recalibration system 106 reconstructs at least some of the call model generated metrics that are not stored within the sample sequencing file(s) provided but were utilized by the call generation model. For instance, as shown in FIG. 6, the call recalibration system 106 determines (i.e., derives) at least some of the call model generated metrics from alternative information within the sequencing data file(s) to determine reconstructed call model generated metrics. For example, in some implementations, the call recalibration system 106 reconstructs certain call model generated metrics, such as the hidden Markov model (HMM) statistics utilized by the call generation model, from other information within the sequencing data files, such as Concise Idiosyncratic Gapped Alignment Report (CIGAR) string output or other sequencing information.

In some cases, the sample sequencing data file(s) 620 have a corresponding ground truth variant call file 616 associated with them, where the ground truth variant call file 616 indicates an actual genotype call and its various metrics that result from the sample sequencing metrics 604. For instance, the call recalibration system 106 utilizes ground truth variant call files from a training dataset from the food and drug administration, called the PrecisionFDA dataset. In some cases, the sample sequencing metrics 604 include a subset of sample sequencing metrics for each genotype call in a ground truth variant call file 616. The ground truth variant call file 616 can have a ground truth variant call (e.g., genotype metric in a genotype field) and/or a ground truth base call corresponding to each subset of sample sequencing metrics.

As further illustrated in FIG. 6, the call recalibration system 106 generates predicted variant-call classifications 608 based on the extracted sample sequencing metrics 604. Specifically, the call recalibration system 106 utilizes a call-recalibration-machine-learning model 606 to generate the predicted variant-call classifications 608. Indeed, in some embodiments, the call-recalibration-machine-learning model 606 generates a set of three predicted variant-call classifications 608 including a predicted false-positive probability, a predicted zygosity-error probability, and a predicted true-positive classification. The predicted variant-call classifications 608 can accordingly take the form of any of the variant-call classifications described above.

Based on the predicted variant-call classifications 608, the call recalibration system 106 determines genotype calls and generates a modified variant call file 610 comprising the modified or updated genotype calls and corresponding fields. As indicated above, the call recalibration system 106 can utilize (i) existing genotype calls generated by a call generation model and included in the sample sequencing data file(s) 620 and (ii) the call-recalibration-machine-learning model 606 to modify data fields corresponding to a variant call file (e.g., of the sample sequencing data file(s) 620) for the genotype call. Such modified or recalibrated values are output in the modified variant call file 610 by, for example the call-recalibration-machine-learning model 606. For example, the call recalibration system 106 determines recalibrated values for particular metrics within the modified variant call file 610, including a base-call-quality metric (QUAL), a genotype metric (GT), and a genotype-quality metric (GQ).

As further illustrated in FIG. 6, the call recalibration system 106 performs a comparison 612. Specifically, the call recalibration system 106 performs the comparison 612 between (i) variant genotype calls and/or data fields in the modified variant call file 610 and (ii) variant genotype calls and/or data fields in the ground truth variant call file 616. In some embodiments, the call recalibration system 106 utilizes a loss function 614 to compare variant genotype calls and/or data fields from the two variant call files (e.g., to determine an error or a measure of loss between them). For instance, in cases where the call-recalibration-machine-learning model 606 is an ensemble of gradient boosted trees, the call recalibration system 106 utilizes a mean squared error loss function (e.g., for regression) and/or a logarithmic loss function (e.g., for classification) as the loss function 614.

By contrast, in embodiments where the call-recalibration-machine-learning model 606 is a neural network, the call recalibration system 106 can utilize a cross entropy loss function, an L1 loss function, or a mean squared error loss function as the loss function 614. For example, the call recalibration system 106 utilizes the loss function 614 to determine a difference between variant genotype calls and/or data fields from the modified variant call file 610 and the ground truth variant call file 616.

As further illustrated in FIG. 6, the call recalibration system 106 performs model fitting 618. In particular, the call recalibration system 106 fits the call-recalibration-machine-learning model 606 based on the comparison 612. For instance, the call recalibration system 106 performs modifications or adjustments to the call-recalibration-machine-learning model 606 to reduce the measure of loss from the loss function 614 for a subsequent training iteration.

For gradient boosted trees or treelite, for example, the call recalibration system 106 trains the call-recalibration-machine-learning model 606 on the gradients of the errors determined by the loss function 614. For instance, the call recalibration system 106 solves a convex optimization problem (e.g., of infinite dimensions) while regularizing the objective to avoid overfitting. In certain implementations, the call recalibration system 106 scales the gradients to emphasize corrections to under-represented classes (e.g., where there are significantly more true positives than false positive variant calls).

In some embodiments, the call recalibration system 106 adds a new weak learner (e.g., a new boosted tree) to the call-recalibration-machine-learning model 606 for each successive training iteration as part of solving the optimization problem. For example, the call recalibration system 106 finds a feature (e.g., a sequencing metric) that minimizes a loss from the loss function 614 and either adds the feature to the current iteration's tree or starts to build a new tree with the feature.

In addition or in the alternative to gradient boosted decision trees, the call recalibration system 106 trains a logistic regression to learn parameters for generating one or more variant-call classifications such as a true-positive classification. To avoid overfitting, the call recalibration system 106 further regularizes based on hyperparameters such as the learning rate, stochastic gradient boosting, the number of trees, the tree-depth(s), complexity penalization, and L1/L2 regularization.

In embodiments where the call-recalibration-machine-learning model 606 is a neural network, the call recalibration system 106 performs the model fitting 618 by modifying internal parameters (e.g., weights) of the call-recalibration-machine-learning model 606 to reduce the measure of loss for the loss function 614. Indeed, the call recalibration system 106 modifies how the call-recalibration-machine-learning model 606 analyzes and passes data between layers and neurons by modifying the internal network parameters. Thus, over multiple iterations, the call recalibration system 106 improves the accuracy of the call-recalibration-machine-learning model 606.

Indeed, in some cases, the call recalibration system 106 repeats the training process illustrated in FIG. 6 for multiple iterations. For example, the call recalibration system 106 repeats the iterative training by selecting a new set of sequencing metrics for each genotype call along with a corresponding ground truth genotype call in a corresponding ground truth variant call file. The call recalibration system 106 further generates a new set of predicted variant-call classifications for each iteration along with a new modified variant call file. As described above, the call recalibration system 106 also compares a variant genotype calls and/or data fields from the modified variant call file at each iteration with the corresponding variant genotype calls and/or data fields from the corresponding ground truth variant call file and further performs model fitting 618. The call recalibration system 106 repeats this process until the call-recalibration-machine-learning model 606 generates predicted variant-call classifications that result in variant calls that satisfies a threshold measure of loss. In some embodiments, the call recalibration system 106 performs the training process of FIG. 6 for homozygous reference coordinates to update or modify variant calls of these coordinates and to thereby recover false negative variant calls (based on simulating haploid data from diploid data and modifying inputs and outputs of the call-recalibration-machine-learning model 606 as described).

As mentioned above, in certain described embodiments, the call recalibration system 106 provides improvements in computing efficiency over existing sequencing systems. FIG. 7, for example, illustrates a table depicting computing efficiency improvements associated with the call recalibration system in accordance with one or more embodiments.

As previously mentioned, some existing sequencing systems utilizing a machine-learning-model-based variant caller must reprocess raw sequencing data (e.g., nucleotide reads from a BCL file) to generate updated genotype calls for a sample nucleotide sequence (e.g., genomic sample). Further, some existing sequencing systems require computer arrays with hardware accelerators, such as a Field Programmable Gate Array (FPGA), to execute a machine-learning-model-based variant caller. In contrast, as discussed in detail above, the call recalibration system 106 utilizes a call-recalibration-machine-learning model that can operate on any processor to recalibrate existing genotype calls based on sequencing metrics extracted or determined from one or more existing sequencing data files (e.g., sequencing data files generated by a previous generation call generation model)—without re-processing raw sequencing metrics (e.g., nucleotide reads from a BCL file).

As indicated in the table of FIG. 7, in at least one implementation, an existing sequencing system requires an average of 20 minutes per genomic sample to reprocess base call data for the corresponding genomic sample. Such existing sequencing systems require 20 minutes per genomic sample despite implementing a large FPGA-equipped computer with 48 parallel processing units and 256 GB of memory. As also indicated in the table of FIG. 7, in at least one implementations, the call recalibration system 106 processes a genomic sample in an average of 7 minutes per genomic sample to generate a recalibrated sequencing data file utilizing a call-recalibration-machine-learning model. By extracting sequencing metrics for genotype calls from existing sequencing data files—and utilizing a call-recalibration-machine-learning model to generate variant-call classifications that facilitate updating previously generated genotype calls and/or corresponding sequencing metrics—the call recalibration system 106 improves the computer processing speed over 2 times relative to existing sequencing systems performing a same basic task. Rather than limiting the call-recalibration-machine-learning model to an FPGA or other reconfigurable array, the call recalibration system 106 expands implementation to any processor, such as a general purpose CPU array comprising 16 processors and 128 GB of memory shown in FIG. 7. Indeed, as shown by the comparative experimental results provided in FIG. 7, the call recalibration system 106 provides for significant improvements in efficiency over existing sequencing systems.

As mentioned above, in certain described embodiments, the call recalibration system 106 provides improvements in both efficiency and accuracy over existing sequencing systems. FIGS. 8A-8B illustrate bar graphs depicting accuracy improvements associated with the call recalibration system 106 in accordance with one or more embodiments. In particular, FIGS. 8A-8B illustrates comparative experimental results of various sequencing systems running various sample genomic sequences.

For example, FIG. 8A illustrates a bar graph comparing performance in the identification of single nucleotide variants (SNPs) within various genomic datasets (i.e., portions of the HG001-HG007 human genome datasets). The illustrated bar graph depicts results of a previous version of a call generation model (e.g., call generation model 204a) labeled “v3.7.8,” an updated version of a call generation machine learning model (e.g., the updated call generation model 204b) labeled “v4.0.3,” and the call recalibration system 106, labeled “v4.2,” utilizing a call-recalibration-machine-learning model to recalibrate nucleotide base reads generated by the previous version of a call generation model.

As shown in FIGS. 8A and 8B, the call recalibration system 106 outperforms the previous call generation model, resulting in fewer false positives (FP) and false negatives (FN) when identifying SNPs within each genome dataset. Moreover, the call recalibration system 106 performs similarly to the updated call generation model utilizing a call-recalibration-machine-learning model whilst improving efficiency thereover (e.g., as discussed above in relation to FIG. 7). Also, the call recalibration system 106 provides similar results when utilizing alternative formats of sequencing data files. As shown, the FP and FN results for SNP calls are similar when utilizing BAM files versus the results of utilizing CRAM files.

In similar fashion as FIG. 8A, FIG. 8B illustrates a bar graph comparing performance in the identification of variants comprising insertions or deletions (Indels) within various genomic datasets (i.e., portions of the HG001-HG007 human genome datasets). The illustrated bar graph depicts results of a previous version of a call generation model (e.g., call generation model 204a) labeled “v3.7.8,” an updated version of a call generation machine learning model (e.g., the updated call generation model 204b) labeled “v4.0.3,” and the call recalibration system 106, labeled “v4.2,” utilizing a call-recalibration-machine-learning model to recalibrate nucleotide base reads generated by the previous version of a call generation model.

As shown, the call recalibration system 106 outperforms the previous call generation model, resulting in fewer false positives (FP) and false negatives (FN) when identifying indels within each genome dataset. Moreover, the call recalibration system 106 performs similarly to the updated call generation model utilizing a call-recalibration-machine-learning model whilst improving efficiency thereover (e.g., as discussed above in relation to FIG. 7). Also, the call recalibration system 106 provides similar results when utilizing alternative formats of sequencing data files. As shown, the FP and FN results for indel calls are similar when utilizing BAM files as input for determining sequencing metrics versus the results of utilizing CRAM files as input for determining sequencing metrics.

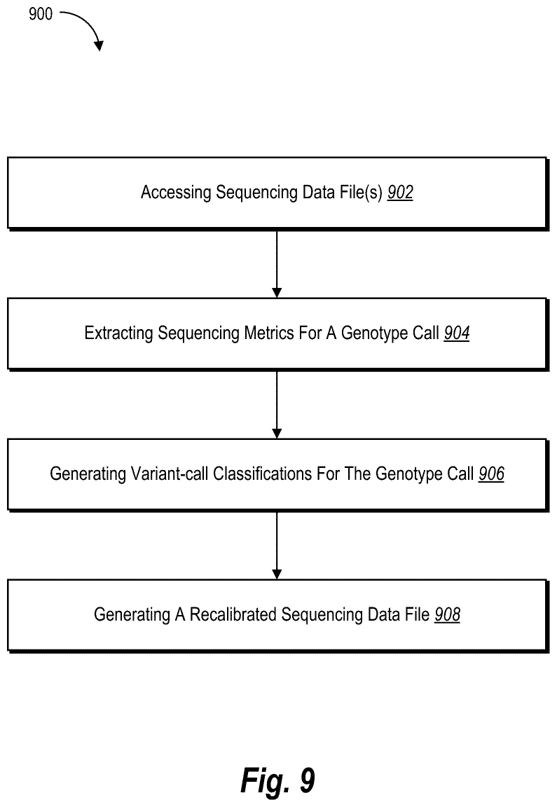

Turning now to FIG. 9, this figure illustrates an example flowchart of a series of acts of generating a recalibrated sequencing data file with an updated genotype call or variant call based on variant-call classifications from a call-recalibration-machine-learning model in accordance with one or more embodiments. While FIG. 9 illustrates acts according to one embodiment, alternative embodiments may omit, add to, reorder, and/or modify any of the acts shown in FIG. 9. The acts of FIG. 9 can be performed as part of a method. Alternatively, a non-transitory computer readable storage medium can comprise instructions that, when executed by one or more processors, cause a computing device to perform the acts depicted in FIG. 9. In still further embodiments, a system comprising at least one processor and a non-transitory computer readable medium comprising instructions that, when executed by one or more processors, cause the system to perform the acts of FIG. 9.

As shown in FIG. 9, the series of acts 900 includes an act 902 of accessing sequencing data file(s), an act 904 of extracting sequencing metrics for a genotype call, an act 906 for generating variant-call classifications for the genotype call, and an act 908 for generating a recalibrated sequencing data file. For example, the series of acts 900 can include acts to perform any of the operations described in the following clauses:

CLAUSE 1. A method comprising:
accessing, for a sample nucleotide sequence, one or more sequencing data files comprising data for nucleotide reads and a genotype call at a genomic coordinate;
extracting, from the one or more sequencing data files, sequencing metrics for the nucleotide reads or the genotype call;
generating, utilizing a call-recalibration-machine-learning model and based on the sequencing metrics, one or more variant-call classifications indicating an accuracy of the genotype call within the one or more sequencing data files; and
generating, based on the one or more variant-call classifications, a recalibrated sequencing data file comprising an updated genotype call at the genomic coordinate for the sample nucleotide sequence.

CLAUSE 2. The method of clause 1, wherein generating the one or more variant-call classifications comprises generating the one or more variant-call classifications without utilizing a call-generation model to contemporaneously generate the genotype call.

CLAUSE 3. The method of any of clauses 1-2, wherein extracting the sequencing metrics for the genotype call comprises extracting one or more read-based sequencing metrics from an alignment data file of the one or more sequencing data files.

CLAUSE 4. The method of any of clauses 1-3, wherein extracting the sequencing metrics comprises extracting one or more read-based sequencing metrics or call-model-generated sequencing metrics for the genotype call from a genotype-call data file of the one or more sequencing data files, the genotype-call data file comprising the genotype call.

CLAUSE 5. The method of any of clauses 1-4, further comprising accessing the genotype-call data file by accessing a variant call format (VCF) file or a genomic variant call format (gVCF) file comprising variant and non-variant calls.

CLAUSE 6. The method of any of clauses 1-5, wherein the genotype-call data file comprising the genotype call was generated on a computing device executing a hardware accelerator and the recalibrated sequencing data file is generated utilizing a general-purpose processing unit as the at least one processor of the system.

CLAUSE 7. The method of any of clauses 1-6, wherein the hardware accelerator comprises a field-programmable gate array (FPGA) or an application specific integrated circuit (ASIC) and the recalibrated sequencing data file is generated utilizing one or more of a central processing unit (CPU) or a graphical processing unit (GPU).

CLAUSE 8. The method of any of clauses 1-7, further comprising:
accessing an additional genotype-call data file generated by a different version of a call-generation model than a version of the call-generation model that generated the genotype-call data file;
extracting, from the additional genotype-call data file, additional sequencing metrics for an additional genotype call at a genomic coordinate for an additional sample nucleotide sequence;
generating, utilizing the call-recalibration-machine-learning model and based on the additional sequencing metrics for the additional genotype call, one or more additional variant-call classifications indicating an accuracy of the additional genotype call within the additional genotype-call data file; and
generating, based on the one or more additional variant-call classifications, an additional recalibrated sequencing data file comprising an updated additional genotype call at the genomic coordinate for the additional sample nucleotide sequence.

CLAUSE 9. The method of any of clauses 1-8, further comprising generating the one or more variant-call classifications by generating one or more of a false-positive probability that the genotype call is a false positive, a genotype-error probability that a genotype for the genotype call is incorrect, or a true-positive probability that the genotype call is a true positive.

CLAUSE 10. The method of any of clauses 1-9, further comprising determining the updated genotype call by:
identifying the genomic coordinate as a multiallelic genomic coordinate;
generating, utilizing the call-recalibration-machine-learning model, the one or more variant-call classifications comprising one or more of a reference probability that the genotype call comprises a homozygous reference genotype at the multiallelic genomic coordinate, a zygosity-error probability that the genotype call comprises a genotype-zygosity error at the multiallelic genomic coordinate, or a true-positive variant probability that the genotype call constitutes a true positive variant at the multiallelic genomic coordinate; and
determining the updated genotype call at the multiallelic genomic coordinate based on one or more of the reference probability, the zygosity-error probability, or the true-positive variant probability.

CLAUSE 11. The method of any of clauses 1-10, further comprising:
modifying, based on the one or more variant-call classifications, one or more of a base-call-quality metric, a genotype-probability metric, a genotype metric, a genotype-likelihood metric, or a genotype-quality metric for the genotype call; and
generating the recalibrated sequencing data file comprising the modified base-call-quality metric, the modified genotype-probability metric, the modified genotype metric, the modified genotype-likelihood metric, or the modified genotype-quality metric.

CLAUSE 12. The method of any of clauses 1-11, further comprising generating, as part of the recalibrated sequencing data file, the updated genotype call at a biallelic genomic coordinate for the sample nucleotide sequence by:
determining a homozygous-reference genotype call at the genomic coordinate instead of a heterozygous-variant genotype call or a homozygous-variant genotype call reported in the one or more sequencing data files;
determining the heterozygous-variant genotype call at the genomic coordinate instead of the homozygous-reference genotype call or the homozygous-variant genotype call reported in the one or more sequencing data files; or
determining the homozygous-variant genotype call at the genomic coordinate instead of the heterozygous-variant genotype call or the homozygous-reference genotype call reported in the one or more sequencing data files.

CLAUSE 13. The method of any of clauses 1-12, further comprising:
extracting, from the one or more sequencing data files, sequencing metrics for an additional genotype call at an additional genomic coordinate for the sample nucleotide sequence;
generating, utilizing the call-recalibration-machine-learning model and based on the sequencing metrics for the additional genotype call, one or more additional variant-call classifications indicating an accuracy of the additional genotype call within the one or more sequencing data files;
modifying, based on the one or more additional variant-call classifications, a base-call-quality metric for the additional genotype call to generate a modified base-call-quality metric that falls below a base-call-quality threshold; and
annotating the additional genotype call to indicate the modified base-call-quality metric falls below the base-call-quality threshold.

CLAUSE 14. The method of any of clauses 1-13, further comprising:
extracting, from the one or more sequencing data files, sequencing metrics for an additional genotype call at an additional genomic coordinate for the sample nucleotide sequence;
generating, utilizing the call-recalibration-machine-learning model and based on the sequencing metrics for the additional genotype call, one or more additional variant-call classifications indicating an accuracy of the additional genotype call within the one or more sequencing data files; and
confirming, based on the one or more additional variant-call classifications, the genotype call at the additional genomic coordinate for the sample nucleotide sequence.

CLAUSE 15. The method of any of clauses 1-14, further comprising training the call-recalibration-machine-learning model by:
generating a plurality of recalibrated sequencing data files from a plurality of sequencing data files corresponding to a plurality of known genomes;
comparing updated genotype calls from the plurality of recalibrated sequencing data files with known variants of the plurality of known genomes; and
adjusting parameters of the call-recalibration-machine-learning model based on differences between the updated genotype calls and the known variants.

CLAUSE 16. A method comprising:
accessing, for a sample nucleotide sequence, one or more sequencing data files comprising a genotype call at a genomic coordinate;
extracting, from the one or more sequencing data files, sequencing metrics for the genotype call;
generating, utilizing a call-recalibration-machine-learning model and based on the sequencing metrics, one or more variant-call classifications indicating an accuracy of the genotype call within the one or more sequencing data files; and
generating, based on the one or more variant-call classifications, a recalibrated sequencing data file comprising an updated genotype call at the genomic coordinate for the sample nucleotide sequence.

CLAUSE 17. The method of clause 16, further comprising:
accessing an alignment data file of the one or more sequencing data files, the alignment data file comprising nucleotide reads corresponding to the genomic coordinate for the sample nucleotide sequence; and
extracting, from the alignment data file, one or more read-based sequencing metrics of the sequencing metrics, the one or more read-based sequencing metrics corresponding to the nucleotide reads.

CLAUSE 18. The method of any of clauses 16-17, further comprising generating the one or more variant-call classifications without utilizing a call-generation model to contemporaneously generate the genotype call.

CLAUSE 19. The method of any of clauses 16-18, wherein extracting the sequencing metrics comprises extracting one or more read-based sequencing metrics or call-model-generated sequencing metrics for the genotype call from a genotype-call data file of the one or more sequencing data files, the genotype-call data file comprising the genotype call.

CLAUSE 20. The method of any of clauses 16-19, further comprising accessing the genotype-call data file by accessing a variant call format (VCF) file or a genomic variant call format (gVCF) file comprising variant and non-variant calls.

CLAUSE 21. The method of any of clauses 16-20, wherein the genotype-call data file comprising the genotype call was generated on a computing device executing a hardware accelerator and the recalibrated sequencing data file is generated utilizing a general-purpose processing unit as the at least one processor of the system.

CLAUSE 22. The method of any of clauses 16-21, wherein the hardware accelerator comprises a field-programmable gate array (FPGA) or an application specific integrated circuit (ASIC) and the recalibrated sequencing data file is generated utilizing one or more of a central processing unit (CPU) or a graphical processing unit (GPU).

CLAUSE 23. The method of any of clauses 16-22, further comprising:
accessing an additional genotype-call data file generated by a different version of a call-generation model than a version of the call-generation model that generated the genotype-call data file;
extracting, from the additional genotype-call data file, additional sequencing metrics for an additional genotype call at a genomic coordinate for an additional sample nucleotide sequence;
generating, utilizing the call-recalibration-machine-learning model and based on the additional sequencing metrics for the additional genotype call, one or more additional variant-call classifications indicating an accuracy of the additional genotype call within the additional genotype-call data file; and
generating, based on the one or more additional variant-call classifications, an additional recalibrated sequencing data file comprising an updated additional genotype call at the genomic coordinate for the additional sample nucleotide sequence.

CLAUSE 24. The method of any of clauses 16-23, further comprising generating the one or more variant-call classifications by generating one or more of a false-positive probability that the genotype call is a false positive, a genotype-error probability that a genotype for the genotype call is incorrect, or a true-positive probability that the genotype call is a true positive.

CLAUSE 25. The method of any of clauses 16-24, further comprising determining the updated genotype call by:
identifying the genomic coordinate as a multiallelic genomic coordinate;
generating, utilizing the call-recalibration-machine-learning model, the one or more variant-call classifications comprising one or more of a reference probability that the genotype call comprises a homozygous reference genotype at the multiallelic genomic coordinate, a zygosity-error probability that the genotype call comprises a genotype-zygosity error at the multiallelic genomic coordinate, or a true-positive variant probability that the genotype call constitutes a true positive variant at the multiallelic genomic coordinate; and
determining the updated genotype call at the multiallelic genomic coordinate based on one or more of the reference probability, the zygosity-error probability, or the true-positive variant probability.

CLAUSE 26. The method of any of clauses 16-25, further comprising:
modifying, based on the one or more variant-call classifications, one or more of a base-call-quality metric, a genotype-probability metric, a genotype metric, a genotype-likelihood metric, or a genotype-quality metric for the genotype call; and
generating the recalibrated sequencing data file comprising the modified base-call-quality metric, the modified genotype-probability metric, the modified genotype metric, the modified genotype-likelihood metric, or the modified genotype-quality metric.

CLAUSE 27. The method of any of clauses 16-26, further comprising generating, as part of the recalibrated sequencing data file, the updated genotype call at a biallelic genomic coordinate for the sample nucleotide sequence by:
determining a homozygous-reference genotype call at the genomic coordinate instead of a heterozygous-variant genotype call or a homozygous-variant genotype call reported in the one or more sequencing data files;
determining the heterozygous-variant genotype call at the genomic coordinate instead of the homozygous-reference genotype call or the homozygous-variant genotype call reported in the one or more sequencing data files; or
determining the homozygous-variant genotype call at the genomic coordinate instead of the heterozygous-variant genotype call or the homozygous-reference genotype call reported in the one or more sequencing data files.

CLAUSE 28. The method of any of clauses 16-27, further comprising:
extracting, from the one or more sequencing data files, sequencing metrics for an additional genotype call at an additional genomic coordinate for the sample nucleotide sequence;
generating, utilizing the call-recalibration-machine-learning model and based on the sequencing metrics for the additional genotype call, one or more additional variant-call classifications indicating an accuracy of the additional genotype call within the one or more sequencing data files;
modifying, based on the one or more additional variant-call classifications, a base-call-quality metric for the additional genotype call to generate a modified base-call-quality metric that falls below a base-call-quality threshold; and
annotating the additional genotype call to indicate the modified base-call-quality metric falls below the base-call-quality threshold.

CLAUSE 29. The method of any of clauses 16-28, further comprising:
extracting, from the one or more sequencing data files, sequencing metrics for an additional genotype call at an additional genomic coordinate for the sample nucleotide sequence;
generating, utilizing the call-recalibration-machine-learning model and based on the sequencing metrics for the additional genotype call, one or more additional variant-call classifications indicating an accuracy of the additional genotype call within the one or more sequencing data files; and
confirming, based on the one or more additional variant-call classifications, the genotype call at the additional genomic coordinate for the sample nucleotide sequence.

CLAUSE 30. The method of any of clauses 16-29, further comprising training the call-recalibration-machine-learning model by:
generating a plurality of recalibrated sequencing data files from a plurality of sequencing data files corresponding to a plurality of known genomes;
comparing updated genotype calls from the plurality of recalibrated sequencing data files with known variants of the plurality of known genomes; and
adjusting parameters of the call-recalibration-machine-learning model based on differences between the updated genotype calls and the known variants.

CLAUSE 31. The method of any of clauses 16-30, wherein:
generating the one or more variant-call classifications comprises generating the variant-call classifications for one or more candidate insertions or deletions (indels) utilizing the call-recalibration-machine-learning model trained with indel training data; and
generating the recalibrated sequencing data file comprises generating, based on the one or more variant-call classifications for the one or more candidate indels, the updated genotype call indicating a presence or absence of an indel at the genomic coordinate for the sample nucleotide sequence.

CLAUSE 32. The method of any of clauses 16-31, wherein:
generating the one or more variant-call classifications comprises generating the variant-call classifications for one or more candidate single nucleotide variants (SNVs) utilizing the call-recalibration-machine-learning model trained with SNV training data; and
generating the recalibrated sequencing data file comprises generating, based on the one or more variant-call classifications for the one or more candidate SNVs, the updated genotype call indicating a presence or absence of a SNV at the genomic coordinate for the sample nucleotide sequence.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Embodiments in which images are obtained in different color channels, for example, coinciding with different labels used to distinguish one nucleotide base type from another are particularly applicable. In some embodiments, the process to determine the nucleotide sequence of a target nucleic acid (i.e., a nucleic acid polymer) can be an automated process. Preferred embodiments include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as release of pyrophosphate; or the like. In embodiments, where two or more different nucleotides are present in a sequencing reagent, the different nucleotides can be distinguishable from each other, or alternatively, the two or more different labels can be the indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred embodiments include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to incorporation of a nucleotides at the features of the array. An image can be obtained after the array is treated with a particular nucleotide type (e.g., A, T, C or G). Images obtained after addition of each nucleotide type will differ with regard to which features in the array are detected. These differences in the image reflect the different sequence content of the features on the array. However, the relative locations of each feature will remain unchanged in the images. The images can be stored, processed and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.), and is also described in WO 91/06678 and WO 07/123,744, each of which is incorporated herein by reference. The availability of fluorescently labeled terminators in which both the termination can be reversed, and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing embodiments, the labels do not substantially inhibit extension under SBS reaction conditions. However, the detection labels can be removable, for example, by cleavage or degradation. Images can be captured following incorporation of labels into arrayed nucleic acid features. In particular embodiments, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially, and an image of the array can be obtained between each addition step. In such embodiments, each image will show nucleic acid features that have incorporated nucleotides of a particular type. Different features are present or absent in the different images due the different sequence content of each feature. However, the relative position of the features will remain unchanged in the images. Images obtained from such reversible terminator-SBS methods can be stored, processed and analyzed as set forth herein. Following the image capture step, labels can be removed, and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular embodiments some or all of the nucleotide monomers can include reversible terminators. In such embodiments, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102: 5932-7 (2005), which is incorporated herein by reference in its entirety). Ruparel et al described the development of reversible terminators that used a small 3′ allyl group to block extension but could easily be deblocked by a short treatment with a palladium catalyst. The fluorophore was attached to the base via a photocleavable linker that could easily be cleaved by a 30 second exposure to long wavelength UV light. Thus, either disulfide reduction or photocleavage can be used as a cleavable linker. Another approach to reversible termination is the use of natural termination that ensues after placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100900, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some embodiments can utilize detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on presence of their respective signals and incorporation of the fourth nucleotide type into the nucleic acid can be determined based on absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary embodiment that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some embodiments can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due the different sequence content of each feature, but the relative position of the features will remain unchanged in the images. Images obtained from ligation-based sequencing methods can be stored, processed and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some embodiments can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such embodiments, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed and analyzed as set forth herein. In particular, the data can be treated as an image in accordance with the exemplary treatment of optical images and other images that is set forth herein.

Some embodiments can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed and analyzed as set forth herein.

Some SBS embodiments include detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular embodiments, different target nucleic acids can be treated in a common reaction vessel or on a surface of a particular substrate. This allows convenient delivery of sequencing reagents, removal of unreacted reagents and detection of incorporation events in a multiplex manner. In embodiments using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as, bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines and the like. A flow cell can be configured and/or used in an integrated system for detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing embodiment as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic acid polymers present in samples received by a sequencing device. As defined herein, “sample” and its derivatives, is used in its broadest sense and includes any specimen, culture and the like that is suspected of including a target. In some embodiments, the sample comprises DNA, RNA, PNA, LNA, chimeric or hybrid forms of nucleic acids. The sample can include any biological, clinical, surgical, agricultural, atmospheric or aquatic-based specimen containing one or more nucleic acids. The term also includes any isolated nucleic acid sample such a genomic DNA, fresh-frozen or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample and normal tissue sample, or sample from a single source that contains two distinct forms of genetic material such as maternal and fetal DNA obtained from a maternal subject, or the presence of contaminating bacterial DNA in a sample that contains plant or animal DNA. In some embodiments, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another embodiment, low molecular weight material includes enzymatically or mechanically fragmented DNA. The sample can include cell-free circulating DNA. In some embodiments, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some embodiments, the sample can be an epidemiological, agricultural, forensic or pathogenic sample. In some embodiments, the sample can include nucleic acid molecules obtained from an animal such as a human or mammalian source. In another embodiment, the sample can include nucleic acid molecules obtained from a non-mammalian source such as a plant, bacteria, virus or fungus. In some embodiments, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one embodiment, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example derived from a buccal swab, paper, fabric or other substrate that may be impregnated with saliva, blood, or other bodily fluids. As such, in some embodiments, the nucleic acid sample may comprise low amounts of, or fragmented portions of DNA, such as genomic DNA. In some embodiments, target sequences can be present in one or more bodily fluids including but not limited to, blood, sputum, plasma, semen, urine and serum. In some embodiments, target sequences can be obtained from hair, skin, tissue samples, autopsy or remains of a victim. In some embodiments, nucleic acids including one or more target sequences can be obtained from a deceased animal or human. In some embodiments, target sequences can include nucleic acids obtained from non-human DNA such a microbial, plant or entomological DNA. In some embodiments, target sequences or amplified target sequences are directed to purposes of human identification. In some embodiments, the disclosure relates generally to methods for identifying characteristics of a forensic sample. In some embodiments, the disclosure relates generally to human identification methods using one or more target specific primers disclosed herein or one or more target specific primers designed using the primer design criteria outlined herein. In one embodiment, a forensic or human identification sample containing at least one target sequence can be amplified using any one or more of the target-specific primers disclosed herein or using the primer criteria outlined herein.

The components of the call recalibration system 106 can include software, hardware, or both. For example, the components of the call recalibration system 106 can include one or more instructions stored on a computer-readable storage medium and executable by processors of one or more computing devices (e.g., the client device 108). When executed by the one or more processors, the computer-executable instructions of the call recalibration system 106 can cause the computing devices to perform the bubble detection methods described herein. Alternatively, the components of the call recalibration system 106 can comprise hardware, such as special purpose processing devices to perform a certain function or group of functions. Additionally, or alternatively, the components of the call recalibration system 106 can include a combination of computer-executable instructions and hardware.

Furthermore, the components of the call recalibration system 106 performing the functions described herein with respect to the call recalibration system 106 may, for example, be implemented as part of a stand-alone application, as a module of an application, as a plug-in for applications, as a library function or functions that may be called by other applications, and/or as a cloud-computing model. Thus, components of the call recalibration system 106 may be implemented as part of a stand-alone application on a personal computing device or a mobile device. Additionally, or alternatively, the components of the call recalibration system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 10 illustrates a block diagram of a computing device 1000 that may be configured to perform one or more of the processes described above. One will appreciate that one or more computing devices such as the computing device 1000 may implement the call recalibration system 106 and the sequencing system 104. As shown by FIG. 10, the computing device 1000 can comprise a processor 1002, a memory 1004, a storage device 1006, an I/O interface 1008, and a communication interface 1010, which may be communicatively coupled by way of a communication infrastructure 1012. In certain embodiments, the computing device 1000 can include fewer or more components than those shown in FIG. 10. The following paragraphs describe components of the computing device 1000 shown in FIG. 10 in additional detail.

In one or more embodiments, the processor 1002 includes hardware for executing instructions, such as those making up a computer program. As an example, and not by way of limitation, to execute instructions for dynamically modifying workflows, the processor 1002 may retrieve (or fetch) the instructions from an internal register, an internal cache, the memory 1004, or the storage device 1006 and decode and execute them. The memory 1004 may be a volatile or non-volatile memory used for storing data, metadata, and programs for execution by the processor(s). The storage device 1006 includes storage, such as a hard disk, flash disk drive, or other digital storage device, for storing data or instructions for performing the methods described herein.

The I/O interface 1008 allows a user to provide input to, receive output from, and otherwise transfer data to and receive data from computing device 1000. The I/O interface 1008 may include a mouse, a keypad or a keyboard, a touch screen, a camera, an optical scanner, network interface, modem, other known I/O devices or a combination of such I/O interfaces. The I/O interface 1008 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1008 is configured to provide graphical data to a display for presentation to a user. The graphical data may be representative of one or more graphical user interfaces and/or any other graphical content as may serve a particular implementation.

The communication interface 1010 can include hardware, software, or both. In any event, the communication interface 1010 can provide one or more interfaces for communication (such as, for example, packet-based communication) between the computing device 1000 and one or more other computing devices or networks. As an example, and not by way of limitation, the communication interface 1010 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1010 may facilitate communications with various types of wired or wireless networks. The communication interface 1010 may also facilitate communications using various communication protocols. The communication infrastructure 1012 may also include hardware, software, or both that couples components of the computing device 1000 to each other. For example, the communication interface 1010 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

We claim:

1. A system comprising:
at least one processor; and
a non-transitory computer readable medium storing instructions that, when executed by the at least one processor, cause the system to:
access, for a sample nucleotide sequence, one or more sequencing data files comprising a genotype call at a genomic coordinate;
extract, from the one or more sequencing data files, sequencing metrics for the genotype call;
generate, utilizing a call-recalibration-machine-learning model and based on the sequencing metrics, one or more variant-call classifications indicating an accuracy of the genotype call within the one or more sequencing data files; and
generate, based on the one or more variant-call classifications, a recalibrated sequencing data file comprising an updated genotype call at the genomic coordinate for the sample nucleotide sequence.

2. The system of claim 1, further comprising instructions that, when executed by the at least one processor, cause the system to:
access an alignment data file of the one or more sequencing data files, the alignment data file comprising nucleotide reads corresponding to the genomic coordinate for the sample nucleotide sequence; and
extract, from the alignment data file, one or more read-based sequencing metrics of the sequencing metrics, the one or more read-based sequencing metrics corresponding to the nucleotide reads.

3. The system of claim 1, further comprising instructions that, when executed by the at least one processor, cause the system to generate the one or more variant-call classifications without utilizing a call-generation model to contemporaneously generate the genotype call.

4. The system of claim 1, wherein extracting the sequencing metrics comprises extracting one or more read-based sequencing metrics or call-model-generated sequencing metrics for the genotype call from a genotype-call data file of the one or more sequencing data files, the genotype-call data file comprising the genotype call.

5. The system of claim 4, further comprising instructions that, when executed by the at least one processor, cause the system to access the genotype-call data file by accessing a variant call format (VCF) or a genomic variant call format (gVCF) file comprising variant and non-variant calls.

6. The system of claim 4, wherein the genotype-call data file comprising the genotype call was generated on a computing device executing a hardware accelerator and the recalibrated sequencing data file is generated utilizing a general-purpose processing unit as the at least one processor of the system.

7. The system of claim 6, wherein the hardware accelerator comprises a field-programmable gate array (FPGA) or an application specific integrated circuit (ASIC) and the recalibrated sequencing data file is generated utilizing one or more of a central processing unit (CPU) or a graphical processing unit (GPU).

8. The system of claim 4, further comprising instructions that, when executed by the at least one processor, cause the system to:
access an additional genotype-call data file generated by a different version of a call-generation model than a version of the call-generation model that generated the genotype-call data file;
extract, from the additional genotype-call data file, additional sequencing metrics for an additional genotype call at a genomic coordinate for an additional sample nucleotide sequence;
generate, utilizing the call-recalibration-machine-learning model and based on the additional sequencing metrics for the additional genotype call, one or more additional variant-call classifications indicating an accuracy of the additional genotype call within the additional genotype-call data file; and
generate, based on the one or more additional variant-call classifications, an additional recalibrated sequencing data file comprising an updated additional genotype call at the genomic coordinate for the additional sample nucleotide sequence.

9. The system of claim 1, further comprising instructions that, when executed by the at least one processor, cause the system to generate the one or more variant-call classifications by generating one or more of a false-positive probability that the genotype call is a false positive, a genotype-error probability that a genotype for the genotype call is incorrect, or a true-positive probability that the genotype call is a true positive.

10. A non-transitory computer readable medium storing instructions that, when executed by at least one processor, cause a system to:
access, for a sample nucleotide sequence, one or more sequencing data files comprising a genotype call at a genomic coordinate;
extract, from the one or more sequencing data files, sequencing metrics for the genotype call;
generate, utilizing a call-recalibration-machine-learning model and based on the sequencing metrics, one or more variant-call classifications indicating an accuracy of the genotype call within the one or more sequencing data files; and
generate, based on the one or more variant-call classifications, a recalibrated sequencing data file comprising an updated genotype call at the genomic coordinate for the sample nucleotide sequence.

11. The non-transitory computer readable medium of claim 10, further storing instructions that, when executed by the at least one processor, cause the system to:
access an alignment data file of the one or more sequencing data files, the alignment data file comprising nucleotide reads corresponding to the genomic coordinate for the sample nucleotide sequence; and
extract, from the alignment data file, one or more read-based sequencing metrics of the sequencing metrics, the one or more read-based sequencing metrics corresponding to the nucleotide reads.

12. The non-transitory computer readable medium of claim 10, further storing instructions that, when executed by the at least one processor, cause the system to determine the updated genotype call by:
identifying the genomic coordinate as a multiallelic genomic coordinate;
generating, utilizing the call-recalibration-machine-learning model, the one or more variant-call classifications comprising one or more of a reference probability that the genotype call comprises a homozygous reference genotype at the multiallelic genomic coordinate, a zygosity-error probability that the genotype call comprises a genotype-zygosity error at the multiallelic genomic coordinate, or a true-positive variant probability that the genotype call constitutes a true positive variant at the multiallelic genomic coordinate; and
determining the updated genotype call at the multiallelic genomic coordinate based on one or more of the reference probability, the zygosity-error probability, or the true-positive variant probability.

13. The non-transitory computer readable medium of claim 10, further storing instructions that, when executed by the at least one processor, cause the system to:
modify, based on the one or more variant-call classifications, one or more of a base-call-quality metric, a genotype-probability metric, a genotype metric, a genotype-likelihood metric, or a genotype-quality metric for the genotype call; and
generate the recalibrated sequencing data file comprising the modified base-call-quality metric, the modified genotype-probability metric, the modified genotype metric, the modified genotype-likelihood metric, or the modified genotype-quality metric.

14. The non-transitory computer readable medium of claim 10, further storing instructions that, when executed by the at least one processor, cause the system to generate, as part of the recalibrated sequencing data file, the updated genotype call at a biallelic genomic coordinate for the sample nucleotide sequence by:
determining a homozygous-reference genotype call at the genomic coordinate instead of a heterozygous-variant genotype call or a homozygous-variant genotype call reported in the one or more sequencing data files;
determining the heterozygous-variant genotype call at the genomic coordinate instead of the homozygous-reference genotype call or the homozygous-variant genotype call reported in the one or more sequencing data files; or
determining the homozygous-variant genotype call at the genomic coordinate instead of the heterozygous-variant genotype call or the homozygous-reference genotype call reported in the one or more sequencing data files.

15. The system of claim 10, further comprising instructions that, when executed by the at least one processor, cause the system to:
extract, from the one or more sequencing data files, sequencing metrics for an additional genotype call at an additional genomic coordinate for the sample nucleotide sequence;
generate, utilizing the call-recalibration-machine-learning model and based on the sequencing metrics for the additional genotype call, one or more additional variant-call classifications indicating an accuracy of the additional genotype call within the one or more sequencing data files;
modify, based on the one or more additional variant-call classifications, a base-call-quality metric for the additional genotype call to generate a modified base-call-quality metric that falls below a base-call-quality threshold; and
annotate the additional genotype call to indicate the modified base-call-quality metric falls below the base-call-quality threshold.

16. A method comprising:
accessing, for a sample nucleotide sequence, one or more sequencing data files comprising a genotype call at a genomic coordinate;
extracting, from the one or more sequencing data files, sequencing metrics for the genotype call;
generating, utilizing a call-recalibration-machine-learning model and based on the sequencing metrics, one or more variant-call classifications indicating an accuracy of the genotype call within the one or more sequencing data files; and
generating, based on the one or more variant-call classifications, a recalibrated sequencing data file comprising an updated genotype call at the genomic coordinate for the sample nucleotide sequence.

17. The method of claim 16, further comprising:
extracting, from the one or more sequencing data files, sequencing metrics for an additional genotype call at an additional genomic coordinate for the sample nucleotide sequence;
generating, utilizing the call-recalibration-machine-learning model and based on the sequencing metrics for the additional genotype call, one or more additional variant-call classifications indicating an accuracy of the additional genotype call within the one or more sequencing data files; and
confirming, based on the one or more additional variant-call classifications, the genotype call at the additional genomic coordinate for the sample nucleotide sequence.

18. The method of claim 16, further comprising training the call-recalibration-machine-learning model by:
generating a plurality of recalibrated sequencing data files from a plurality of sequencing data files corresponding to a plurality of known genomes;
comparing updated genotype calls from the plurality of recalibrated sequencing data files with known variants of the plurality of known genomes; and
adjusting parameters of the call-recalibration-machine-learning model based on differences between the updated genotype calls and the known variants.

19. The method of claim 16, wherein:
generating the one or more variant-call classifications comprises generating the one or more variant-call classifications for one or more candidate insertions or deletions (indels) utilizing the call-recalibration-machine-learning model trained with indel training data; and
generating the recalibrated sequencing data file comprises generating, based on the one or more variant-call classifications for the one or more candidate indels, the updated genotype call indicating a presence or absence of an indel at the genomic coordinate for the sample nucleotide sequence.

20. The method of claim 16, wherein:
generating the one or more variant-call classifications comprises generating the one or more variant-call classifications for one or more candidate single nucleotide variants (SNVs) utilizing the call-recalibration-machine-learning model trained with SNV training data; and
generating the recalibrated sequencing data file comprises generating, based on the one or more variant-call classifications for the one or more candidate SNVs, the updated genotype call indicating a presence or absence of a SNV at the genomic coordinate for the sample nucleotide sequence.
